SEQUENCE LISTING (2 sequences)

SEQ ID NO: 1 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELAGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 2 - protein, 298 aa, Homo sapiens
MSDIEEVVEEYEEEEQEEAAVEEEEDWREDEDEQEEAAEEDAEAEAETEETRAEEDEEEEEAKEAEDGPMEESKPKPRSFMPNLVPPKIPDGERVDFDDIHRKRMEKDLNELQALIEAHFENRKKEEEELVSLKDRIERRRAERAEQQRIRNEREKERQNRLAEERARREEEENRRKAEDEARKKKALSNMMHFGGYIQKQAQTERKSGKRQTEREKKKKILAERRKVLAIDHLNEDQLREKAKELWQSIYNLEAEKFDLQEKFKQQKYEINVLRNRINDNQKVSKTRGKAKVTGRWK

(19) United States
(12) Patent Application Publication
(10) Pub. No.: US 2018/0284134 A1
(43) Pub. Date: Oct. 4, 2018

(54) DETECTION OF ACUTE MYOCARDIAL INFARCTION

(71) Applicant: CMC CONSULTING BOSTON, INC., Framingham, MA (US)

(72) Inventors: Giacomo Basadonna, Framingham, MA (US); Michal RACIBORSKI, Framingham, MA (US); Deepak; SAHU, Framingham, MA (US)

(21) Appl. No.: 15/761,717

(22) PCT Filed: Sep. 21, 2016
(86) PCT No.: PCT/US16/52894

§ 371 Date: Mar. 20, 2018

Related U.S. Application Data
(60) Provisional application No. 62/232,327, filed on Sep. 24, 2015.

(51) Int. Cl. G01N 33/68; G01N 33/53; A61B 5/0452

(52) U.S. Cl. CPC G01N 33/6893; G01N 33/53; C07K 14/4716; G01N 2333/4712; A61B 5/0452

(57) ABSTRACT

In accordance with some embodiments herein, methods, systems, and kits provides for detection and/or treatment of acute myocardial infarction (AMI). In some embodiments, the methods, systems, and kits can comprise a combination of one or more high-sensitivity cardiac troponin (hsCTn) and conventional cardiac troponin (cCTn) tests. In some embodiments, a determination of a presence or absence of AMI is made within a six-hour phase after the subject presents with acute coronary symptoms.

### CROSS-REFERENCE TO RELATED APPLICATIONS

The present application claims the benefit of U.S. Provisional Application No. 62/232,327, filed Sep. 24, 2015, entitled “Detection of Acute Myocardial Infarction,” which is hereby incorporated by reference in its entirety.

### SEQUENCE LISTING

The present application is being filed along with a Sequence Listing in electronic format. The Sequence Listing is provided as a file entitled CMCCB001WO.txt, created Sep. 20, 2016, which is 4746 bytes in size. The information in the electronic format of the Sequence Listing is incorporated herein by reference in its entirety.

### BACKGROUND

Acute coronary syndrome (ACS) is a major cause of morbidity and mortality around the world. The most common manifestation of ACS is acute myocardial infarction (AMI). Early detection and treatment of AMI can have a major impact on AMI morbidity and mortality and therefore on associated costs. Acute MI is part of a group of conditions collectively known as acute coronary syndrome, which includes ST-segment-elevation myocardial infarction (STEMI), NSTEMI, and unstable angina. These conditions are associated with common symptoms but have different underlying pathologies. STEMI is usually associated with a relatively large amount of damage to the myocardium (heart muscle) caused by a major blockage in the coronary artery, and can be detected from ST-elevation on an electrocardiogram (ECG) trace. By comparison, NSTEMI is often associated with relatively less severe damage to the myocardium, caused by either partial blockage of the coronary artery or blockage of a smaller artery, and does not produce ST-elevation on ECG. Acute coronary syndromes arise from an obstruction in the coronary arteries, usually caused by atherosclerosis, a build-up of plaque, which ruptures. When blood flow to the heart is reduced or blocked for a significant length of time (around 30-60 minutes), damage to cardiomyocytes (heart muscle cells) occurs. This is a pathological change that distinguishes an acute MI from unstable angina.

Cardiac troponin I and cardiac troponin T are biological markers of cardiac muscle death (cardiomyocyte necrosis). They are released into the circulation when damage to cardiac muscle has occurred. Troponins C, I and T form the troponin-tropomyosin complex which is responsible for regulating cardiac muscle contraction.

It is desirable, as quickly as possible, to accurately diagnose AMI so that patients who have experienced AMI will quickly receive the appropriate life-saving treatments for AMI, which can be invasive, sometimes risky, and expensive, and so that the patients who have not experienced AMI can be released from care or can be given other treatments in accordance with their conditions, which can diminish the inconvenience to the patients and the expense for payers of health care costs.

### FIELD

Embodiments herein relate to methods, systems, and/or kits for medical diagnosis and treatment. In particular, some embodiments herein relate to methods, systems, and/or kits involving a combination of conventional and high-sensitivity troponin tests for the detection and, if detected, treatment of Acute Myocardial Infarction (AMI) within six hours of when a subject presents with acute coronary symptoms.

### SUMMARY

In accordance with some embodiments herein, methods, systems, and kits are provided for detection and/or treatment of acute myocardial infarction (AMI). In some embodiments, the methods, systems, and kits can comprise a combination of one or more high-sensitivity cardiac troponin (hsCTn) and conventional cardiac troponin (cCTn) tests. In some embodiments, a determination of a presence or absence of AMI is made within a six-hour phase after the subject presents with acute coronary symptoms. Most, if not all, of the results described herein are based upon predictions of clinical results, based in some cases on data from actual tests performed for other purposes.

### BRIEF DESCRIPTION OF THE DRAWINGS

FIG. 1 is a flow diagram illustrating approaches for the initial assessment on presentation at ED with Chest Pain.

FIG. 2 is a flow diagram illustrating a general flow of patients in cardiac troponin testing. FIG. 2 is provided as two panels, in which FIG. 2 represents the left-hand portion of the figure, and FIG. 2 (cont'd) represents the right-hand portion.

FIG. 3 is a flow diagram summarizing methods of detecting acute myocardial infarction (AMI) and/or treating a subject for AMI in accordance with some embodiments herein.

FIG. 4A is a schematic diagram illustrating the exterior of a system for determining AMI in accordance with some embodiments herein.

FIG. 4B is a schematic diagram illustrating a cutaway of a system for determining AMI in accordance with some embodiments herein.

FIGS. 5A and 5B together are a flow diagram illustrating an example of predictive results of a determination of AMI for 1000 candidate patients comprising methods in accordance with some embodiments herein. FIG. 5A represents the first portion of the flow diagram (“phases 1-3”) and FIG. 5B represents the second portion of the flow diagram (“phases 4-5”).

FIG. 6 is a schematic diagram illustrating a system for determining AMI in accordance with some embodiments herein.

FIG. 7 is a graph illustrating an example system of sensitivities of cardiac troponin assays in accordance with some embodiments herein. The graph of FIG. 7 provides a schematic representation of the effect of increasing cTnI sensitivity relative to a healthy population and the measured 99th percentile for each example class of assay, along with 10% and 20% CV limits. It is noted that the indicated ranges are provided by way of example. For example, the indicated “ideal” high sensitivity range reflects a balance between selectivity and sensitivity, and while this range of sensitivity can be suitable for hsCTn assays in accordance with some embodiments herein, other ranges are also suitable for hsCTn assays in some embodiments. In some embodiments, suitable ranges for any of these tests can be performed between approximately any of the values provided in the graph of FIG. 7. In some embodiments, suitable ranges for any of these tests can be performed between any of the values provided in the graph of FIG. 7.

### DETAILED DESCRIPTION

In accordance with some embodiments herein, methods, devices, and kits are provided for determining a presence, absence, and/or risk of AMI in a subject who presents with acute coronary symptoms. In some embodiments, a plurality of different tests with differing sensitivities and specificities can be performed in one or more sequences at a plurality of time intervals to determine whether a subject has or has not experienced AMI. For example, a high-sensitivity cardiac troponin (hsCTn) test and a conventional cardiac troponin (cCTn) test can be applied to samples from a subject obtained in one or more sequences at a plurality of time intervals, such as within about six hours of when the subject presents with acute coronary symptoms. Without being limited by any theory, it is contemplated that a hsCTn test provides a high sensitivity, and thus minimizes a possibility of false negatives, while a cCTn tests provides a high specificity, and thus diminishes a possibility of false positives. Accordingly, a combination of hsCTn tests and cCTn tests at intervals in accordance with some embodiments herein can accurately and detect AMI for samples obtained within a six-hour phase of when the subject presents with acute coronary symptoms.

In some embodiments, the sensitivity and/or selectivity of the detection of CTn in samples is tuned at different phases after a patient presents with acute coronary symptoms so as to detect AMI with minimal likelihood of false positive and minimal likelihood of false negatives. Without being limited by any theory, it is contemplated that the use of hsCTn and cCTn tests in sequences in accordance with some embodiments herein can balance selectivity and sensitivity. It is noted that both hsCTn and cCTn tests can detect cardiac troponin (e.g. troponin I), but have different cutoffs for sensitivity and selectivity. As such, in some embodiment, the tuning of sensitivity and selectivity can be accomplished by performing a sequence of hsCTn and cCTn tests as described herein.

Troponins I and T can be used as biomarkers for diagnosing myocardial infarction (MI), for which a rise and fall in troponin levels can signify that myocardial damage has occurred. The optimum sensitivity of conventional troponin assays for Acute Myocardial Infarction (AMI) is understood to occur 10-12 hours after the onset of symptoms (see, e.g. the NICE UK guidelines for the use of biochemical markers). For this reason many patients undergo hospital admission and observation while serial troponin testing is carried out. In view of the timeline for cCTn assays, hsCTn assays have been developed. These hsCTn are able to detect lower levels of troponin in the blood and also within few hours of onset of symptoms as compared to conventional troponin assays, leading to improved early detection of AMI. However, high-sensitivity cardiac troponin owing to its relatively low specificity was believed to possess substantial challenges in term of high number of false positive cases.

Without being limited by any theory, it is contemplated that within a population of patients, there can be variations in levels of biological markers. For this reason, when making a diagnosis or determination based on levels of a biological marker, there is a trade-off between sensitivity and specificity. For example, a diagnostic test or determination can be very sensitive, so as to minimize false negatives, but as a consequence of the high sensitivity, can also exhibit lower specificity, resulting in false positives. For example, a diagnosis or determination can be highly specific, so as to minimize false positives, but at the expense of sensitivity, resulting in false negatives. Thus, a quantitative or qualitative result from a biological marker test by itself does not necessarily provide an accurate assessment from which a diagnosis or determination can be made, as there is a possibility of a false positive or false negative, depending on the specificity and sensitivity of the test.

It is noted that cCTn tests are understood to be less sensitive than hsCTn tests, and cCTn tests are typically not implemented within six hours of when a subject presents with acute coronary symptoms. Without being limited by any theory, it has been believed that cCTn tests are not sensitive enough to detect levels and/or changes in cardiac troponin within six hours of when a subject presents with these symptoms. Rather, some approaches for cCTn tests include an initial assessment of cardiac troponin (I or T) levels 6-12 hours after the onset of symptoms. In accordance with some embodiments herein, cCTn can be used for samples obtained from a subject at or before a six-hour phase of when a subject presents with acute coronary symptoms, and can offer a high level of specificity for such samples.

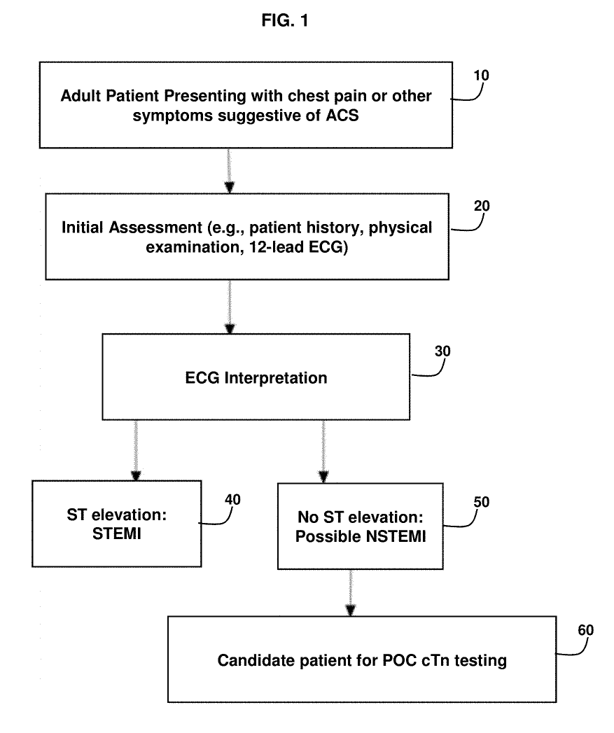

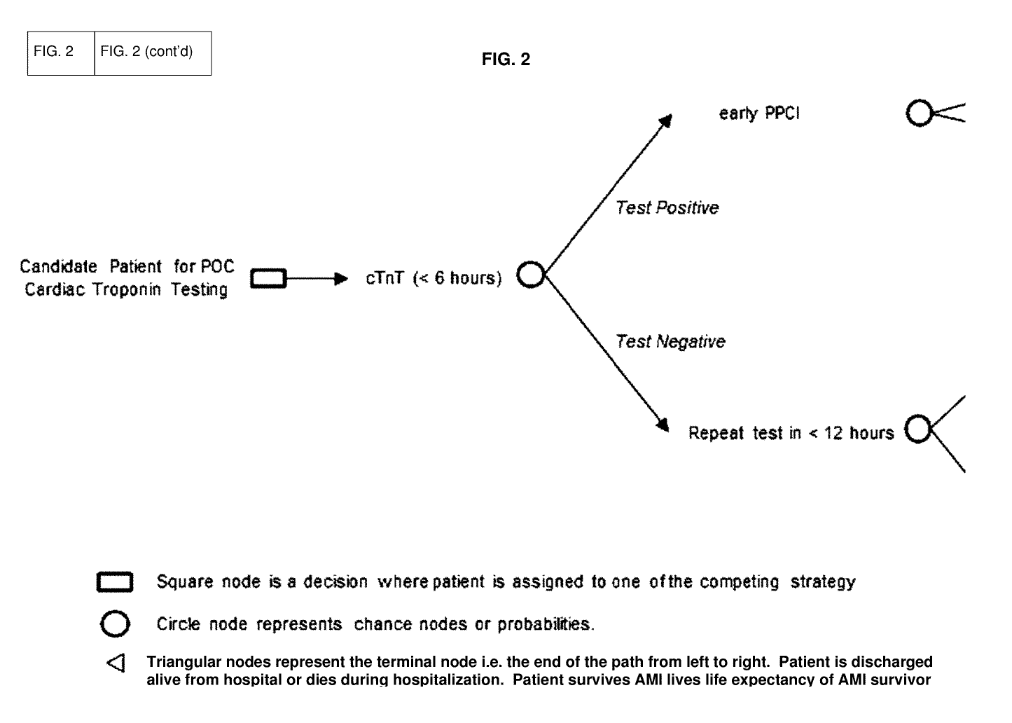

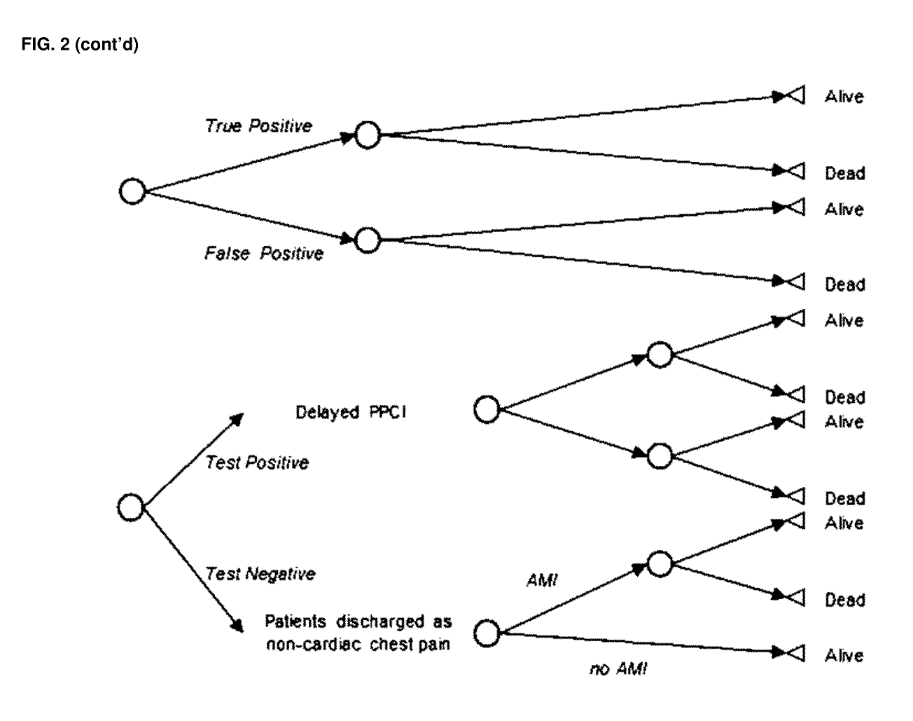

In accordance with some embodiments herein, patients with acute coronary syndrome often present with acute chest pain and other symptoms such as nausea, vomiting, dyspnoea, sweating and indigestion. As used herein “acute coronary symptoms” refer to any recognized outward symptoms of AMI, such as acute chest pain, and one or more of nausea, vomiting, dyspnoea, sweating and indigestion. These symptoms, including acute chest pain, are common to many other conditions such as anxiety, gastro-oesophageal reflux disease and muscle strain. An initial assessment for such subjects is illustrated in the flow diagrams of FIG. 1. An adult patient can present with chest pain or other symptoms suggestive of ACS. 10. An initial assessment (e.g., patient history, physical examination, 12-lead ECG) can be made 20. There can be an ECG interpretation. 30. If there is ST elevation, a determination of STEMI can be made. 40. If there is no ST elevation, there is possible NSTEMI. 50. Such a patient with possible NSTEMI can be a candidate for POC cTn testing. 60. An initial assessment can include taking a resting 12-lead ECG along with a clinical history, a physical examination and biochemical marker analysis, and managing patients in whom regional ST-segment elevation or presumed new left branch bundle block is observed on ECG according to Myocardial infarction with ST-segment elevation. Patients without persistent ST-elevation changes on ECG can be given a working diagnosis of a suspected non-ST-segment-elevation acute coronary syndrome. Some approaches for the use of biochemical markers for subjects without persistent ST-elevation changes on ECG are summarized in FIG. 2, and may include: (a) Take a blood sample for troponin I or troponin T on initial assessment in hospital (these are preferred biochemical markers to diagnose AMI); (b) Take a second blood sample for troponin I or T measurement 6-12 hours after the onset of symptoms; (c) Do not use biochemical markers such as natriuretic peptides and high sensitivity C-reactive protein to diagnose acute coronary syndrome; (d) Do not use biochemical markers of myocardial ischaemia (such as ischaemia modified albumin) as opposed to markers of necrosis when assessing people with acute chest pain; and (e) Take into account the clinical presentation, the time from onset of symptoms and the resting 12-lead ECG findings, when interpreting troponin measurements.

It is noted that an ECG can typically identify ST-segment-elevation myocardial infarction (STEMI), but that non-ST segment elevation myocardial infarction (NSTEMI) does not produce ST-elevation on ECG, and thus an ECG cannot readily distinguish NSTEMI from other pathologies that result in acute coronary symptoms. Accordingly, in some embodiments herein, methods, systems, and/or kits are used on subjects that present with acute coronary symptoms, but do not exhibit ST-elevation on ECG. In some embodiments, the methods, systems, and/or kits are used on subjects that generally present with acute coronary symptoms (and are not limited by a particular ECG result). As such, in some embodiments, the methods, systems, and kits can be used to distinguish NSTEMI from unstable angina, conditions that need different treatment.

In accordance with some embodiments herein, a combination of conventional cardiac troponin (cCTn) and high-sensitivity (hsCTn) tests is performed within a first phase, such as about a six-hour phase, from when a subject presents with acute coronary symptoms. Without being limited by any theory, it is contemplated that the hsCTn test is highly sensitive, so that a “negative” hsCTn test result is unlikely to be a false negative, but rather provides a high (e.g. at least about: 99%, 99.5%, or 99.9%) likelihood of a true negative. Without being limited by any theory, it is contemplated that a cCTn test within six hours of when the patient presents with symptoms is highly specific, so that a “positive” result is unlikely to be a false positive, but rather provides a high (e.g. at least about: 95%, 96%, 97%, 98%, 99%, 99.5%, or 99.9%) likelihood of a true positive. Accordingly, in accordance with some embodiments herein, the combination of a hsCTn test and a cCTn test on samples taken through a six-hour phase after the subject presents with symptoms can provide a highly sensitive and highly specific result, in which both false positives and false negatives are minimized.

Moreover, methods, systems, and kits in accordance with some embodiments herein employ an iterative approach for determining a presence or absence of AMI (and thus a decision to treat AMI). As such, methods, systems, and kits in accordance with some embodiments herein can positively identify AMI with high accuracy hours ahead of conventional testing approaches for a comparable patient population (e.g., of subjects without persistent ST-elevation changes on ECG), and thus facilitate prompt treatment for AMI so as to minimize further damage from reduced or blocked blood flow to the heart. Accordingly, methods, systems, and kits in accordance with some embodiments herein can rule out AMI hours before conventional approaches for a conventional patient population (of subjects without persistent ST-elevation changes on ECG), and thus can minimize invasive, and potentially risky AMI treatment such as cardiac catheterization (“Cath Lab”) for subjects that are highly unlikely to have AMI.

### Troponin Tests

A “high-sensitivity cardiac troponin (hsCTn)” test is used herein in accordance with its ordinary meaning in the field, and includes an assay for detecting troponin in which (1) total imprecision (CV) at the 99th percentile value is less than or equal to about 10% (“guideline acceptable”), and (2) measurable concentrations below the 99th percentile are attainable with an assay at a concentration value above the assay's limit of detection for at least about 95% of healthy individuals. In some embodiments, a hsCTn test is an assay for detecting cardiac troponin that has a higher sensitivity than a conventional cardiac troponin (cCTn) test.

Example commercially-available hsCTn tests that can be used in accordance with embodiments herein include, but are not limited to the Abbot ARCHITECT™ hsCTn test, the Beckman ACCESS™ hsCTn test, the NanosphereMTP™ hsCTn test, the Singulex ERENNA™ hsCTn test, the Siemens VISTA™ hsCTn test, the Roche hs-TnT hsCTn test, which may also be referred to as the Roche Elecsys™ hsCTn test.

A “conventional cardiac troponin (cCTn)” test is used herein in accordance with its ordinary meaning in the field, and includes a clinically-accepted assay for detecting troponin in which (i) total imprecision (CV) at the 99th percentile value is greater than or equal to about 10%, and/or (ii) measurable concentrations below the 99th percentile are attainable with an assay at a concentration value above the assay's limit of detection for less than or equal to about 50%/o of healthy individuals.

It is further contemplated that in some circumstances, a “medium cardiac troponin” (mCTn) test can be used in any method or device disclosed in this specification instead of or in addition to a hsCTn test, such as when used in conjunction with a cCTn test of sufficiently low sensitivity. As “medium cardiac troponin” (mCTn) test is used herein in accordance with its ordinary meaning in the field, and can include a class of assays for detecting troponin in which total imprecision (CV) at the 99th percentile value is less than or equal to about 20%. In some embodiments, a mCTn test provides lower sensitivity in order to gain some specificity, relative to an hsCTn test. However, if a mCTm test is paired with a cCTn test having a CV at about the 99th percentile greater than or equal to about 20%, and used in conjunction with methods and systems as described herein, it is contemplated that useful information about AMI can be obtained.

A hsCTn test or cCTn test in accordance with some embodiments herein can detect cardiac troponin I, cardiac troponin T, or both cardiac troponin I and cardiac troponin T. In some embodiments, the hsCTn test or cCTn test comprises an antibody or antibody fragment that binds specifically to troponin I or troponin T. The amino acid sequence of troponin I is well known to one skilled in the art, and one skilled in the art can readily obtain an antibody or antibody fragment that binds specifically to cardiac troponin I (by way of example, a sequence of human troponin I, available as UniProt accession no P19429 is provided herewith as SEQ ID NO: 1, MADGSSDAAREPRPAPAPIRRRSS NYRAYATEPHAKKKSKISASRKLQLKTLLLQIAKQELEREAEERRGEKGRALSTRCQ PLELAGLGFAELQDLCRQLHARVDKVDEERYDIEAKVTKNITEIADLTQKIFDLRGKF KRPTLRRVRISADAMMQALLGARAKESLDLRAHLKQVKKEDTEKENREVGDWRKN IDALSGMEGRKKKFES). The amino acid sequence of cardiac troponin T is well known to one skilled in the art, and one skilled in the art can readily obtain an antibody or antibody fragment that binds specifically to cardiac troponin T (by way of example, a sequence of human troponin T, available as UniProt accession no P45379 is provided herewith as SEQ ID NO: 2, MSDIEEVVEEYEEEEQEEAAVEEEEDWREDEDEQEEAAEEDAEAEAETEE TRAEEDEEEEEAKEAEDGPMEESKPKPRSFMPNLVPPKIPDGERVDFDDIHRKRMEK DLNELQALIEAHFENRKKEEEELVSLKDRIERRRAERAEQQRIRNEREKERQNRLAEE RARREEEENRRKAEDEARKKKALSNMMHFGGYIQKQAQTERKSGKRQTEREKKKK ILAERRKVLAIDHLNEDQLREKAKELWQSIYNLEAEKFDLQEKFKQQKYEINVLRNR INDNQKVSKTRGKAKVTGRWK).

For hsCTn tests and/or cCTn that comprise an antibody or fragment thereof, a number of immunoassay test formats are available in accordance with some embodiments herein. Example suitable immunoassay formats include, but are not limited to, lateral flow assays, no-wash assays, enzyme-linked immunosorbent assay (ELISA, for example sandwich ELISA, competition ELISA, and the like), quantitative western blots, and the like. Furthermore, in some embodiments, the immunoassay comprises detection at least one of chemiluminescence (for example, “flash” chemiluminescence), fluorescence (for example, via fluorescently-labeled antibody, a fluorescence enzyme immunoassay, and/or time-resolved fluorescence), electrochemical emission detection, immunochromato detection, or spectrophotometric detection. In some embodiments, the hsCTn and/or cCTn test comprises a Chemiluminescence immunoassay (CLEIA). In some embodiments, the hsCTn and/or cCTn test comprises a Fluorescent antibody method (e.g., a sandwich method, such as a sandwich no-wash assay or sandwich ELISA). In some embodiments, the hsCTn and/or cCTn test comprises a fluorescent immunoassay (FIA). In some embodiments, the hsCTn and/or cCTn test comprises a fluorescence enzyme immunoassay (FEIA). In some embodiments, the hsCTn and/or cCTn test comprises a chemiluminescence immunoassay (CLEIA). In some embodiments, the hsCTn and/or cCTn test comprises “flash” chemiluminescence. In some embodiments, the hsCTn and/or cCTn test comprises an electrochemical continuous emission immunoassay (ECLIA). In some embodiments, the hsCTn and/or cCTn test comprises a test according to a fluorescent antibody (immunity) law. In some embodiments, the hsCTn and/or cCTn test comprises a time-resolved fluorescence method, (e.g., a EUROPIUM assay). In some embodiments, the hsCTn and/or cCTn test comprises an immunochromato assay. In some embodiments, the hsCTn and/or cCTn test comprises a spectrophotometric assay. In some embodiments, the hsCTn and/or cCTn test comprises immunonephelometry. In some embodiments, the hsCTn and/or cCTn test comprises a sandwich immunoassay.

A no-wash assay can detect the presence or absence of cardiac troponin through the detection of a signal (or the absence of a signal) indicating the association of two different detectable moieties. By way of example, suitable no-wash assays can comprise a first antibody specific for cardiac troponin (I or T), in which the antibody comprises a first detectable moiety, and a second antibody specific for a different epitope of the same cardiac troponin as the first antibody, in which the second antibody comprises a second detectable moiety. The first and second detectable moiety can comprise a FRET pair, for example a donor moiety and an acceptor moiety, so that the association of the first and second detectable moiety within a FRET radius produces a detectable signal (or absence of a signal that would otherwise be present). In some embodiments, the first and second moieties comprise a flurophore-quencher pair.

By way of example, suitable lateral flow assays in accordance with some embodiments herein comprise a first, labeled antibody specific for cardiac troponin (I or T), and a second antibody specific for the same cardiac troponin (but that binds the cardiac troponin at a different epitope than the first labeled antibody so that both antibodies can bind cardiac troponin simultaneously) that is immobilized on a substrate, for example in a spot or stripe. The first labeled antibody and the second labeled antibody can be connected by a porous material. As such, when the first labeled antibody is contacted with fluid comprising cardiac troponin (I or T), the first labeled antibody can bind to the cardiac troponin, and be carried by the fluid through the porous material to the second antibody, which can capture the troponin bound the first labeled antibody, thus immobilizing the label at the location of the second antibody (e.g. the spot or stripe). Thus, the appearance of label at the location of the second antibody can indicate the presence of the indicated cardiac troponin (I or T). The first labeled antibody can be labeled with a detectable moiety, for example a dye particle, metal particle (e.g. gold particles such as nanoparticles), flurophore, Raman spectroscopy label (e.g. labels for Surface Enhanced Raman Spectroscopy, SERS, for example, metal-comprising particles), and the like. In accordance with some embodiments, one or more troponin tests can be performed on or in, or facilitated with, a test consumable receptacle (e.g., a container or a substrate). Some examples of test consumable receptacles include a cartridge, test tube, test strip, ELISA substrate, lateral flow matrix, or the like, which can comprise reagents for at least one cardiac troponin test (e.g. hsCTn or cCTn). In some embodiments, a single test consumable receptacle can be configured for both an hsCTn and cCTn test. Some test consumable receptacles are configured for hsCTn tests, while other test consumable receptacles are configured for cCTn tests. In some embodiments, the test consumable receptacles are labeled, for example color-coded. The label can identify the particular type of cardiac troponin test that a test consumable receptacle is to be used for, and/or a particular phase it is to be used at. In some embodiments, a test consumable receptacle is labeled with a suitable label to direct a user to use hsCTn and cCTn test consumable receptacles in a suitable sequence in accordance with the methods described herein. For example, the test consumable receptacles can be labeled with sequential numbers, letters, symbols, colors, or the like to instruct a user to run the tests in a sequence in accordance with the methods described herein. For example, in some embodiments a first hsCTn test consumable receptacle is labeled with a “1” to direct the user to use the first hsCTn test consumable receptacle initially, a subsequent cCTn test consumable receptacle is labeled with a “2” to direct the user to use a subsequent hsCTn test consumable receptacle at the one-hour phase, a second cCTn test consumable receptacle is labeled with a “3” to direct the user to use a second hsCTn test consumable receptacle at the three-hour phase, a third hsCTn test consumable receptacle is labeled with a “4” to direct the user to use a third hsCTn test at the four-hour phase, a third hsCTn test consumable receptacle is labeled with a “5” to direct the user to use a third hsCTn test consumable receptacle at the four-hour phase, and a fourth cCTn test consumable receptacle is labeled with a “6” to direct the user to use a fourth cCTn test consumable receptacle at the six-hour phase. These labeling are only illustrated by way of example, and it will be understood that there are numerous suitable labeling schemes to direct a user to use the test consumable receptacles in a suitable sequence. Table 0 illustrates a variety of example labeling schemes suitable for use in accordance with some embodiments herein. Moreover, it will be appreciated that a number of alternative schemes and/or variations to the indicated schemes that also convey a suitable sequence of test consumable receptacles will also be suitable for use in accordance with some embodiments herein.

TABLE 0
Example Labeling Schemes for Test Consumable Receptacles

| | Type of test | Example | Example | Example | Example |
|---|---|---|---|---|---|
| | Consumable | Labeling | Labeling | Labeling | Labeling |
| Phase | Receptacle | Scheme | Scheme | Scheme | Scheme |
| Initial | hsCTn | 1 | A | 0 | * |
| One-hour | hsCTn | 2 | B | 1 | ** |
| Three- | cCTn | 3 | C | 3 | *** |
| hour | | | | | |
| Four-hour | hsCTn | 4 | D | 4a | **** |
| | cCTn (if | 5 | E | 4b | ***** |
| | applicable) | | | | |
| Six-hour | cCTn | 6 | F | 6 | ****** |

It is contemplated that labeling the test consumable receptacles to identify the sequence of hsCTn and cCTn in accordance with the methods as described herein can facilitate the performance of the method, and further, can reduce the likelihood of user error compared to, for example, test consumable receptacles that are labeled with the kind of test, but do not indicate the sequence.

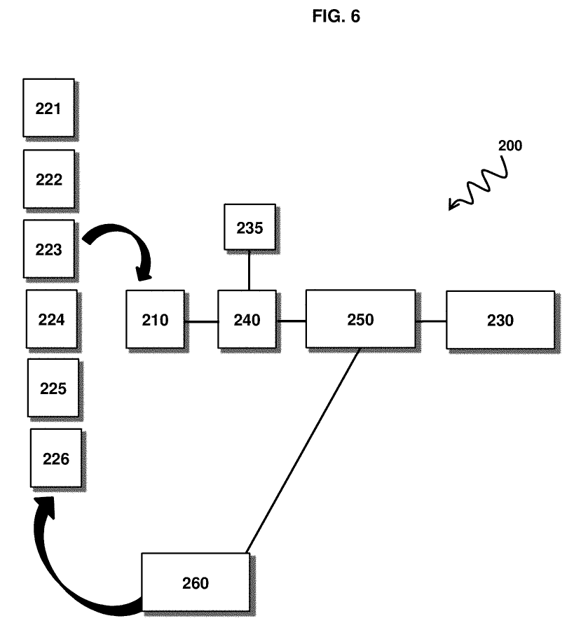

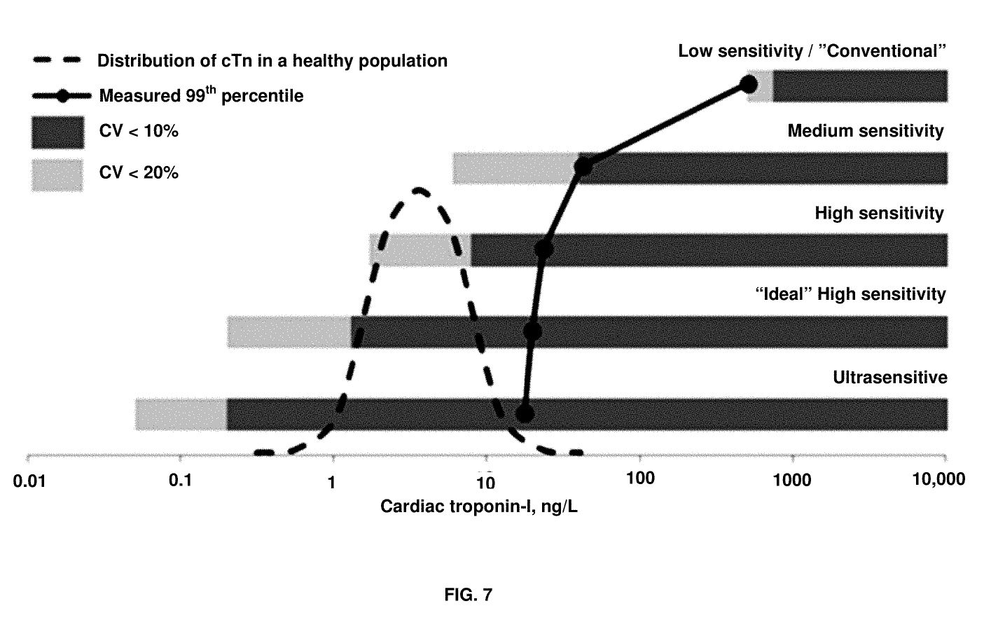

In some embodiments, the test consumable receptacle further comprises a tag that corresponds to the sequence for using hsCTn and cCTn test consumable receptacles in accordance with a method as described herein. The tag can be readable by a system as described herein, for example a bar code or an electronic signal, an RFID, a magnetic key, a character or characterize recognizable by optical character recognition (e.g. letters, numbers, symbols, or a combination thereof), or a non-transient computer memory (e.g. a computer readable medium comprising a flash memory), a physical shape of an object, or an electrical connection provided by an object, or any other suitable tag. In some embodiments, the tag corresponds to a label on the test consumable receptacle that indicates the sequence of hsCTn and cCTn in accordance with the methods as described herein. Accordingly, a system as described herein can read the tag, and if an incorrect kind of test consumable receptacle is inserted for the current phase, the system can display an error message as described herein. In some embodiments, a set of test consumable receptacles for performing are provided in a kit as described herein (e.g. about 3 hsCTn and about 3 cCTn test consumable receptacles in a packaging as described herein), and the test consumable receptacles comprise tags that also identify the test consumable receptacles of the same kit, for example to allow the system to keep track of which test consumables correspond to which patient. Accordingly, the tag can provide information to allow the system to correlate Accordingly, if the method is being performed for two or more patients on the same system at the same time, the tags can allow the system to track each patient (and thus which phase of the method is correct at a given time). Thus, in some embodiments, if incorrect test consumable receptacle for the instant phase, kind of test (hsCTn or cCTn), or patient identifying information in inserted in the system, the system can alert the user, for example via an user alert function and/or structure 235 as described herein (see FIG. 6).

Troponin tests (hsCTn and cCTn) tests in accordance with embodiments herein can be performed on a variety of samples obtained from a patient, for example whole blood, fractions thereof, urine, or saliva. In some embodiments, the sample comprises whole blood. In some embodiments, the sample comprises a fraction of whole blood. It is noted that tests suitable for detecting cardiac troponin in whole blood can involve minimal processing, and are suitable for point-of-care tests. In some embodiments, the troponin test (hsCTn and/or cCTn) is configured to detect cardiac troponin in whole blood. It is noted that workflow can be simplified, and the number of samples collected can be minimized if an embodiment utilizes a pairing of hsCTn and cCTn tests suitable for the same sample type. In some embodiments, the hsCTn test and cCTn test are configured to operate on the same sample type, for example whole blood.

It is noted that in accordance with some embodiments herein, hsCTn and/or cCTn tests can be performed on samples obtained at various time periods after the subject first presents with acute coronary symptoms:

In some embodiments herein, an hsCTn test result is compared to a baseline hsCTn level (referred to herein as “baseline” hsCTn), determined for an “initial sample” obtained from a subject at the time the subject presents with acute coronary symptoms. As used herein an “initial sample” (including pluralizations and variations of this root term), or a sample obtained “at the time the subject presents with acute coronary symptoms” (including pluralizations and variations of this root term) refers to the sample that provides the “baseline” or “time-zero (T₀)” cardiac troponin metrics for cardiac troponin tests performed on later-obtained samples from the subject (e.g. iterative tests in accordance with some embodiments herein), and is taken from the subject at an interval to represent presentation with acute coronary symptoms. An “initial sample” or sample obtained “at the time the subject presents with acute coronary symptoms” need not be taken at the exact moment the subject enters a medical facility (for example an emergency room). For example, an initial sample can be taken while a subject is being transported to undergo care from a medical care provider, as part of an initial patient intake or assessment by a medical care provider, and/or as the first in a series of determinations of cardiac troponin levels in accordance with some embodiments herein even when the sample is not taken at the very first moment that the subject's acute coronary symptoms are observed. Additionally, it is noted that in accordance with some embodiments, the initial sample is not necessarily the absolute first sample taken from a subject, as it is possible that a second, or third, fourth, or subsequent sample taken from a patient could be used as the “initial” sample in a series of cardiac troponin tests.

As used herein, a sample obtained from a subject at the “first phase” such as a “one hour phase” (including pluralizations and variations of this root term) after the subject presents with symptoms of acute coronary disease refers to the collection of the sample at a time understood to be a one-hour follow-up from the collection of the initial sample in the course assessing and/or treating the subject in accordance with the typical standard of care. Accordingly, as used herein, a sample obtained at the “one hour phase” is not limited to a sample taken at exactly one-hour-down-to-the-minute after the collection of the initial sample. Rather, taking a sample at the “one hour phase” encompasses times somewhat less than one hour and somewhat more than one hour, so long as they do not substantively change the diagnostic or clinical outcome under the particular circumstances. In some embodiments, the first phase is about 40 minutes, 45, 50, 55, 60, 65, 70, 75, or 80 minutes after the collection of the initial sample, including ranges between any of the two listed values, for example about 40-80 minutes, about 45-75 minutes, about 50-60 minutes, 50-70 minutes, about 55-65 minutes, about 60-70 minutes, or about 60-80 minutes after. In some embodiments, the one-hour phase is at a time about 55-65 minutes after the collection of the initial sample. In some embodiments, the one-hour phase is at a time about 59-61 minutes after the collection of the initial sample.

As used herein, a sample obtained from a subject at the “second phase” such as at a “three hour phase” (including pluralizations and variations of this root term) after the subject presents with symptoms of acute coronary disease refers to the collection of the sample at a time understood to be a three-hour follow-up from the collection of the initial sample in the course assessing and/or treating the subject in accordance with the typical standard of care. Accordingly, as used herein, a sample obtained at the “three hour phase” is not limited to a sample taken at exactly three-hours-down-to-the-minute after the collection of the initial sample. Rather, taking a sample at the “three hour phase” encompasses times somewhat less than three hours and somewhat more than three hours, so long as they do not substantively change the diagnostic or clinical outcome under the particular circumstances. In some embodiments, the second phase is about 150 minutes, 155, 160, 165, 170, 175, 180, 185, 190, 195, 200, 210, or 220 minutes after the collection of the initial sample, including ranges between any of the two listed values, for example about 160-200 minutes, about 165-195 minutes, about 170-180 minutes, about 170-190 minutes, about 175-185 minutes, about 180-190 minutes, or about 180-200 minutes after. In some embodiments, the three-hour phase is at a time about 175-185 minutes after the collection of the initial sample. In some embodiments, the three-hour phase is at a time about 179-181 minutes after the collection of the initial sample.

As used herein, a sample obtained from a subject at the “third phase” such as at a “four hour phase” (including pluralizations and variations of this root term) after the subject presents with symptoms of acute coronary disease refers to the collection of the sample at a time understood to be a four-hour follow-up from the collection of the initial sample in the course assessing and/or treating the subject in accordance with the typical standard of care. Accordingly, as used herein, a sample obtained at the “four hour phase” is not limited to a sample taken at exactly four-hours-down-to-the-minute after the collection of the initial sample. Rather, taking a sample at the “third phase” encompasses times somewhat less than four hours and somewhat more than four hours, so long as they do not substantively change the diagnostic or clinical outcome under the particular circumstances. In some embodiments, the four hour phase is about 210 minutes, 215, 220, 225, 230, 235, 240, 245, 250, 255, 260, 265, or 270 minutes after the collection of the initial sample, including ranges between any of the two listed values, for example about 220-260 minutes, about 225-255 minutes, about 230-240 minutes, about 230-250 minutes, about 235-245 minutes, about 240-250 minutes, or about 240-260 minutes after. In some embodiments, the four-hour phase is at a time about 235-245 minutes after the collection of the initial sample. In some embodiments, the four-hour phase is at a time about 239-241 minutes after the collection of the initial sample.

As used herein, a sample obtained from a subject at the “fourth phase” such as at a “six hour phase” (including pluralizations and variations of this root term) after the subject presents with symptoms of acute coronary disease refers to the collection of the sample at a time understood to be a six-hour follow-up from the collection of the initial sample in the course assessing and/or treating the subject in accordance with the typical standard of care. Accordingly, as used herein, a sample obtained at the “six hour phase” is not limited to a sample taken at exactly six-hours-down-to-the-minute after the collection of the initial sample. Rather, taking a sample at the “six hour phase” encompasses times somewhat less than six hours and somewhat more than six hours, so long as they do not substantively change the diagnostic or clinical outcome under the particular circumstances. In some embodiments, the fourth phase is about 330 minutes, 335, 340, 345, 350, 355, 360, 365, 370, 375, 380, 385, or 390 minutes after the collection of the initial sample, including ranges between any of the two listed values, for example about 340-380 minutes, about 345-375 minutes, about 350-360 minutes, about 350-370 minutes, about 355-365 minutes, about 360-370 minutes, or about 370-380 minutes after. In some embodiments, the six-hour phase is at a time about 355-365 minutes after the collection of the initial sample. In some embodiments, the six-hour phase is at a time about 359-361 minutes after the collection of the initial sample. In some embodiments, the fourth phase or the six hour phase is the last phase. Any of the phases after the first phase can be referred to as a “subsequent phase” or a “later phase.” Any of the phases after the first phase and before the last phase can be referred to as an “interim phase.”

As used herein a hsCTn test result is referred to as “negative” (or “rule-out”) (including pluralizations and variations of either of these root terms) in accordance with the ordinary meaning within the field, and includes when the test determines a baseline cardiac troponin T level to be less than or equal to about 12 ng/L (for example, less than or equal to about 12 ng/L, 11 ng/L, 10 ng/L, 9 ng/L, 8 ng/L, 7 ng/L, 6 ng/L, 5 ng/L, 4 ng/L, 3 ng/L, 2 ng/L, or 1 ng/L) and also determines an absolute change at the one-hour phase compared to the baseline to be less than or equal to about 3 ng/L (for example, a change of less than or equal to about 3 ng/L, 2 ng/L, 1 ng/L, 0.5 ng/L, or 0.1 ng/L). A “negative” (or “rule-out”) can also be determined when the hsCTn detects a baseline cardiac troponin (CTn) level to be less than or equal to about 5 ng/l, for example, less than or equal to about 5 ng/L, 4 ng/L, 3 ng/L, 2 ng/L, or 1 ng/L, including ranges between any two of the listed values. It is noted that a hsCTn test may determine a level of troponin T directly (e.g. by directly measuring a level of troponin T), or indirectly (e.g. by measuring a level of another marker which is indicative of troponin T levels and/or from which troponin T can be inferred, for example by measuring a level of total cardiac troponin and cardiac troponin I, from which the amount of cardiac troponin T could be inferred from subtraction).

As used herein, a hsCTn test result is referred to as “positive” (or “rule-in”) (including pluralizations and variations of either of these root terms) in accordance with the ordinary meaning within the field, and includes when the test determines a baseline cardiac troponin T level to be greater than or equal to than about 52 ng/L (for example, greater than or equal to about 53 ng/L, 55 ng/L, 60 ng/L, 65 ng/L, 70 ng/L 75 ng/L, 100 ng/L, or 150 ng/L) or an absolute change compared to the baseline of greater than or equal to about 5 ng/L (for example, a change of greater than or equal to about 5 ng/L, 6 ng/L, 7 ng/L, 8 ng/L, 9 ng/L, 10 ng/L, 15 ng/L, 20 ng/L, or 25 ng/L).

As used herein, a hsCTn test result is referred to as “observational” (including pluralizations and variations of this root term) in accordance with the ordinary meaning within the field, and includes when the test result does not fit into the “negative” or “positive” criteria.

It is noted that a hsCTn test result that is “not positive” could be either “negative” or “observational”. It is noted that a hsCTn result that is “not negative” could be either “positive” or “observational”.

As used herein, a cCTn test result is understood to be “positive” (including pluralizations and variations of this root term) in accordance with the ordinary meaning within the field, and includes when a level of troponin T is detected to be greater than or equal to about 0.01 ng/ml (for example, greater than or equal to about 0.01 ng/ml, 0.02 ng/ml, 0.05 ng/ml, 0.1 ng/ml, 0.2 ng/ml, or 0.5 ng/ml). A cCTn test result is understood to be “not positive” (e.g., “negative” or “observational”) (including pluralizations and variations of this root term) in accordance with the ordinary meaning within the field, and includes when the level of troponin T is detected to be less than or equal to about 0.01 ng/ml (for example, less than or equal to about: 0.01 ng/ml, 0.005 ng/ml, 0.002 ng/ml, or 0.001 ng/ml).

As a shorthand, it may be stated herein that the cardiac troponin (hsCTn or cCTn) test is “negative”, “positive”, or “observational”, which will be understood as referring to the indicated test result as negative, positive, or observational.

Methods of Determining AMI and/or Treating a Subject that Present with Acute Coronary Symptoms

In accordance with some embodiments herein, methods of treating a subject that presents with acute coronary symptoms are provided. The method can comprise performing a first high-sensitivity cardiac troponin (hsCTn) test. The first hsCTn test can comprise two steps, an initial hsCTn test and a subsequent hsCTn test. The initial hsCTn test can be performed on an initial sample obtained from the subject at the time the subject presents with acute coronary symptoms. The subsequent hsCTn test on a subsequent sample obtained from the subject at a one-hour phase after the subject presents with acute coronary symptoms, in which, if the first hsCTn test is negative, acute myocardial infarction (AMI) is ruled out (and it will be understood that treatment for AMI is not required). If the first hsCTn test is not negative, the method can comprise performing at least one of: (i) a second conventional cardiac troponin (cCTn) test on a second sample obtained no more than about three hours after the subject presents with acute coronary symptoms; or (ii) a third cCTn test on a third sample obtained from the subject no more than about four hours after the subject presents with acute coronary symptoms. If the second cCTn test or third cCTn test is positive, the method can comprise determining the subject to have AMI, and treating the subject for AMI. In some embodiments, if the first hsCTn test is positive, the second cCTn test can be performed on the second sample. In some embodiments, if the first hsCTn test is observational or if the second cCTn test is observational, the method can further comprise performing the third hsCTn test performed on a third sample obtained from the subject at a four-hour phase after the subject presents with acute coronary symptoms. In some embodiments, if the third hsCTn test is positive, a third cCTn test can be performed on the third sample or on another sample obtained from the subject at the four-hour phase after the subject presents with acute coronary symptoms. If the third hsCTn test is not positive, the third cCTn test is not required. The third hsCTn test can be performed prior to the third cCTn test. If the third hsCTn test is not positive, or if the third cCTn test is not positive, the method can further comprise performing a fourth cCTn test on a fourth sample obtained from the subject at a six-hour phase after the subject presents with acute coronary symptoms. If the fourth cCTn test is negative, the subject can be determined to not have AMI (and it will be understood that no AMI treatment is required for the subject). If the fourth cCTn test is not negative, the subject remains a candidate for conventional 6-12 hour AMI observation. It is noted that for methods in accordance with some embodiments herein, the last category (of the fourth cCTn test being performed and being not negative) is relatively rare, and as illustrated in Example 1, of 1000 patients assessed with methods in accordance with embodiments herein, only 43 (4.3%) fell into this last category, while the remaining 95.7% were categorized as either having or not having AMI.

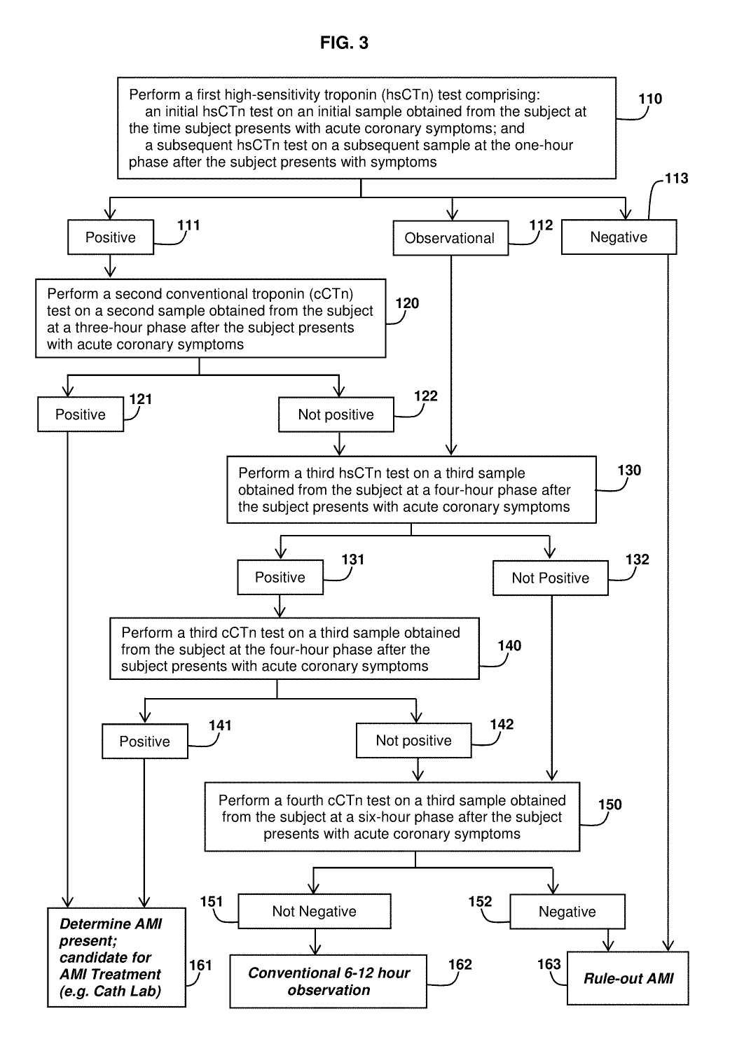

FIG. 3 is a flow diagram depicting a method in accordance with some embodiments herein. The method can comprise performing a first high-sensitivity troponin (hsCTn) test, in which the first hsCTn test comprises: (i) an initial hsCTn test on an initial sample obtained from the subject at the time subject presents with acute coronary symptoms, and (ii) a subsequent hsCTn test on a subsequent sample at the one-hour phase after the subject presents with symptoms 110. The results of the first hsCTn test can be positive 111, observational 112, or negative 113. If the result of the first hsCTn test is positive 111, the method can comprise performing a second conventional troponin (cCTn) test on a second sample obtained from the subject at a three-hour phase after the subject presents with acute coronary symptoms 120. The result of the second cCTn test can be positive 121 or not positive 122. If the result of the second cCTn test is not positive 122 or if the result of the first hsCTn test is observational 112, a third hsCTn test can be performed on a third sample obtained from the subject at a four-hour phase after the subject presents with acute coronary symptoms 130. The results of the third hsCTn test can be positive 131 or not positive 132. If the results of the third hsCTn test are positive 131, a third cCTn test can be performed on a third sample obtained from the subject at the four-hour phase after the subject presents with acute coronary symptoms 140. The third cCTn test can be performed on the same sample as the third hsCTn test, or a different sample from the third hsCTn test (but also collected during at the four-hour phase). The results of the third cCTn test can be positive 141 or not positive 142. If the result of the third hsCTn test is not positive 132, of if the result of the third cCTn test is not positive 142, a fourth cCTn test can be performed on a third sample obtained from the subject for at a six-hour phase after the subject presents with acute coronary symptoms 150. The results of the fourth cCTn test can be not negative 151 or negative 152. If the result of the second cCTn test is positive 121, or if the result of the third cCTn test is positive 141, AMI can be determined to be present 161. For methods comprising diagnosing AMI, AMI treatment of the subject can be proposed or recommended. For methods comprising treatment of AMI, AMI treatment (e.g. Cath lab) of the subject can be performed. If the result of the fourth cCTn test is not negative 151, conventional 6-12 hour observation of the subject can be recommended or proposed 162. For methods comprising treatment of AMI, conventional 6-12 hour observation of the subject can be performed. If the result of the first hsCTn test is negative 113, or if the result of the fourth cCTn test is negative 152, AMI can be ruled-out 163. In some embodiments, if AMI is ruled-out 163, a subject can be discharged. In some embodiments, for example if the method is implemented on a system as described herein, an alarm can be issued to a user (e.g. a health care provider), and/or the test can be disabled or prevented, and/or test results can be discarded or not saved or not used in the diagnostic algorithm, if the user attempts to perform the incorrect kind of cardiac troponin test and/or attempts to perform a particular cardiac troponin test in an incorrect order and/or at an incorrect time. In some embodiments, a kit of test consumable receptacles as described herein are provided to a user performed a method as described herein. The kit of test consumable receptacles can be provided in a pouch, bag, canister, box, blister pack or the like. In some embodiments, the set of test consumable receptacles is provided in a single packaging. In some embodiments, the packaging contains about six consumable receptacles (for example 3 hsCTn test consumable receptacles and 3 cCTn test consumable receptacles, or 4 hsCTn test consumable receptacles and 4 cCTn test consumable receptacles, or 4 hsCTn test consumable receptacles and 3 cCTn test consumable receptacles, or 3 hsCTn test consumable receptacles and 4 cCTn test consumable receptacles). In some embodiments, six test consumable receptacles (3 hsCTn test consumable receptacles and 3 cCTn test consumable receptacles) are provided together in a package, container, bag, or the like, so that a user can use these test consumable receptacles for a method as described herein. In some embodiments, instructions to use the test consumable receptacles in accordance with some embodiments herein are also provided to the user. In some embodiments, the test consumable receptacle comprises a tag which identifies the type of test, phase, and kit from which the test consumable receptacle came from. The tag can be read by a system as described herein, for example to correlate test results with phases of a method being performed for a particular patient, and/or to alert a user if an incorrect test consumable receptacle is being read by system for a given phase and/or patient.

For this and other functions, structures, and processes, disclosed herein, the functions, structures and steps may be implemented or performed in differing order or sequence. Furthermore, the outlined functions and structures are only provided as examples, and some of these functions and structures may be optional, combined into fewer functions and structures, or expanded into additional functions and structures without detracting from the essence of the disclosed embodiments.

It is contemplated that every step of the method outlined in FIG. 3 need not be performed in accordance with every embodiment. To the contrary, any step or combination of steps can be used in appropriate circumstances, either alone or in combination with other embodiments disclosed herein or elsewhere. For example, once an outcome 161, 162, or 163 is reached, additional testing may be skipped. For example, if the result of the first hsCTn test 110 is negative 113, AMI can be ruled-out, and additional testing may be skipped. For example, if the result of the first hsCTn test 110 is positive 112, and the result of the second cCTn test 120 is positive 121, the subject can be determined to have AMI, and can proceed to AMI treatment (e.g. Cath lab) after the sample obtained at the three-hour phase is tested. Consequently, the subject can be treated soon after the test result is obtained, which can lead to superior clinical outcomes with respect to mortality and morbidity compared to conventional approaches that involve waiting a longer period of time before treatment. It is noted that by skipping further testing, resources can be conserved (e.g. the number of test consumable receptacles used can be minimized, the number of samples needed from a subject can be minimized), and the subject can be discharged and/or treated more promptly. For example, if the third hsCTn test 130 is performed before the third cCTn test 140, resources can be conserved in the aggregate, in that if the result of the third hsCTn test is not positive 132, a fourth cCTn test 150 can be performed on a third sample obtained from the subject at a six-hour phase after the subject presents with acute coronary symptoms 150, so that the third cCTn test 140 is not required.

It is contemplated that some steps of the method outlined in FIG. 3 can be performed concurrently in accordance with some embodiments herein. For example, in some embodiments the third hsCTn test 130 and third cCTn test 140 can be performed at the same time. It is contemplated that performing the third hsCTn test 130 and third cCTn test 140 at the same time may be helpful when time is of the essence (as it may arrive at the result faster than performing the two tests serially).

In some embodiments, the third hsCTn test 130 and third cCTn test 140 can be performed on the same sample. In some embodiments, the third hsCTn test 130 and third cCTn test 140 can be performed on different samples.

Without being limited by any theory, it is contemplated that the sensitivity of hsCTn tests, and the specificity of cCTn tests in accordance with some embodiments herein can provide high positive predictive values (PPV) and high negative predictive values. A high PPV represents a strong likelihood that a subject identified as positive for AMI is a true positive (with very few false positives). A high NPV strong likelihood that a subject identified as negative for AMI is a true negative (with very few false negatives). In some embodiments, a method, test, system, or kit in provides a PPV of at least about 900/%, for example at least about 91%, 92%, 93%, 94%, 95%, 96%, 97%, 98%, 99%, 99.5%, or 99.9%, based on a 95% confidence interval. In some embodiments, a method, test, system, or kit in provides an NPV of at least about 90%, for example at least about 91%, 92%, 93%, 94%, 95%, 96%, 97%, 98%, 99%, 99.5%, or 99.9%, based on a 95% confidence interval. In some embodiments, a method, test, system, or kit in provides an NPV of at least about 95%, and a PPV of at least about 95%, based on a 95% confidence interval. In some embodiments, a method, test, system, or kit in provides an NPV of at least about 96%, and a PPV of at least about 96%, based on a 95% confidence interval. In some embodiments, a method, test, system, or kit in provides an NPV of at least about 98%, and a PPV of at least about 98%, based on a 95% confidence interval.

In some embodiments, a method of determining AMI in a subject who presents with acute coronary symptoms is provided. The method can comprise performing a first high-sensitivity cardiac troponin (hsCTn) test comprising: (i) receiving an initial sample obtained from the subject at the time the subject presents with acute coronary symptoms and performing an initial hsCTn test on the initial sample, and (ii) receiving a subsequent sample obtained from the subject at a one-hour phase after the subject presents with acute coronary symptoms and performing a subsequent hsCTn test on the subsequent sample. If the first hsCTn test is negative, the method can comprise determining an absence of AMI. If the first hsCTn test is not negative (e.g. observational or positive), the method can comprise receiving at least one of (i) a second sample obtained from the subject at a four-hour phase after the subject presents with acute coronary symptoms, or (ii) a third sample obtained from the subject at a four-hour phase after the subject presents with acute coronary symptoms, and performing at least one of: (i) a second conventional cardiac troponin (cCTn) test on the second sample; or (ii) a third cCTn test on the third sample. If the second cCTn test or third cCTn test is positive, the method can comprise determining the subject to have AMI. In some embodiments, if the first hsCTn test is positive, the second cCTn test can be performed on the second sample. In some embodiments, if the first hsCTn test is observational or if the second cCTn test is observational, the method can further comprise performing the third hsCTn test performed on a third sample obtained from the subject at a four-hour phase after the subject presents with acute coronary symptoms. In some embodiments, if the third hsCTn test is positive, a third cCTn test can be performed on the third sample or on another sample obtained from the subject at the four-hour phase after the subject presents with acute coronary symptoms. If the third hsCTn test is not positive, the third cCTn test is not required. The third hsCTn test can be performed prior to the third cCTn test. If the third hsCTn test is not positive, or if the third cCTn test is not positive, the method can further comprise performing a fourth cCTn test on a fourth sample obtained from the subject at a six-hour phase after the subject presents with acute coronary symptoms. If the fourth cCTn test is negative, the subject can be determined to not have AMI. If the fourth cCTn test is not negative, the subject can be determined to be observational, and the subject can be determined to possibly have AMI and can be recommended for conventional 6-12 hour AMI observation. It is noted that this latter category of subjects determined to possibly have AMI (rather than those determined to have or not have AMI) is relatively rare, and as outlined in Example 1, only 4.3% of a population of 1000 patients were determined to possibly have AMI, while 95.7% were determined to have a presence of AMI or an absence of AMI. In some embodiments, the method further comprises steps and/or sequences outlined in FIG. 3.

In some embodiments, a method or device as described anywhere in this specification is performed using a mCTn test instead of or in addition to a hsCTn test, such as in conjunction with a cCTn that has a CV at about the 99th percentile value that is greater than or equal to about 20%.

### Systems

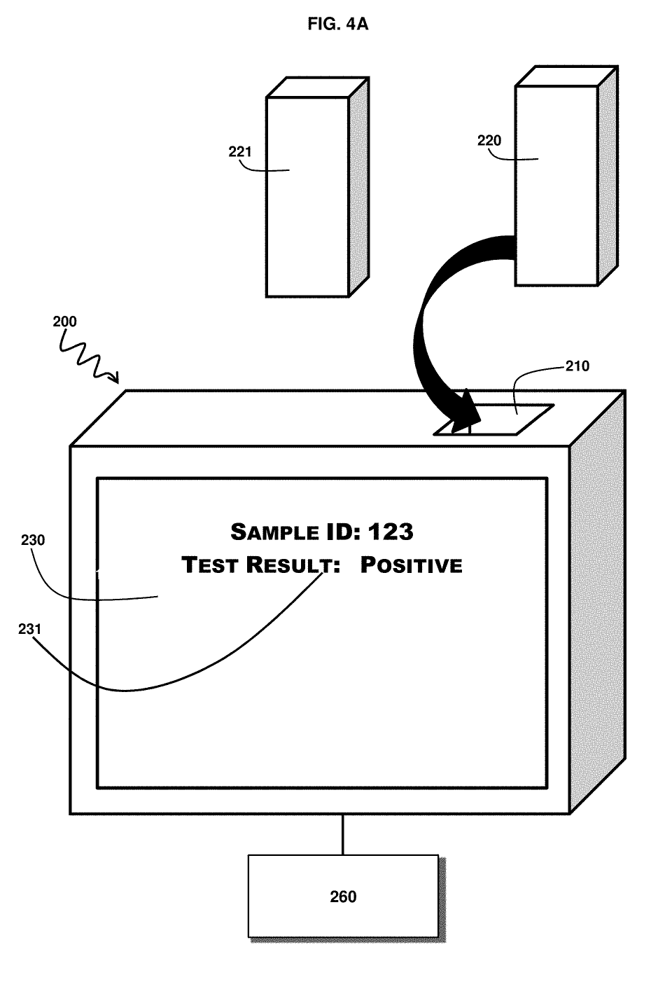

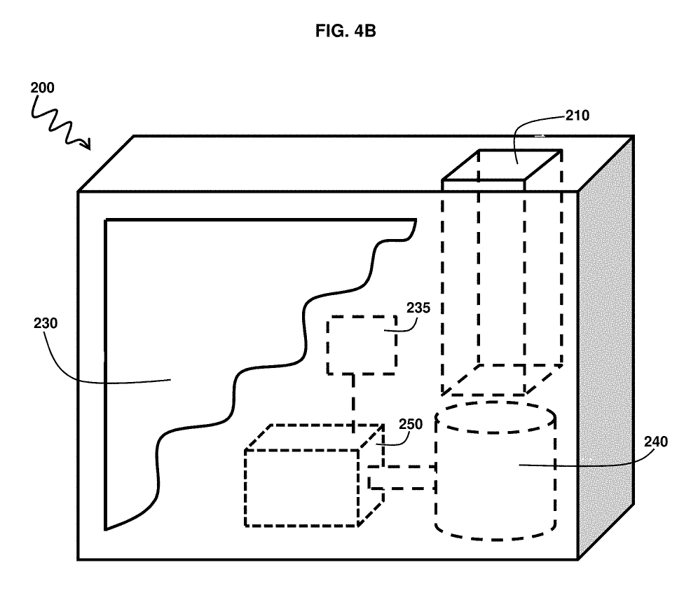

FIGS. 4A, 4B, and 6 are schematic diagrams depicting an example system 200 in accordance with some embodiments herein. The system can be for determining a presence or absence of AMI in a subject. The system 200 can be used to determine a presence or absence of AMI according to methods described herein, for example the methods depicted in FIG. 3. FIG. 4A depicts an exterior view of the example system 200. FIG. 4B depicts a cutaway of the exterior of the system to more readily show additional features. FIG. 6 depicts the components of the system, which can be packaged in any of a number of form factors as described herein. The system 200 can comprise a port 210 configured to receive a test consumable receptacle 221, 222, 223, 224, 225, 226 for a hsCTn or cCTn test. In some embodiments, the port 210 is configured to read a tag on the test consumable receptacle 221, 222, 223, 224, 225, 226 as described herein, for example so that the system can ascertain the type of test, phase of test, and/or kit that the test came from (which in turn can identify a particular patient). For example, the port can comprise a barcode reader, RFID reader, magnetic reader, electrical reader, optical character recognition scanner, wired or wireless data port, or any other suitable reader so as to read the tag of the test consumable receptacle. In some embodiments, the system comprises a single port 210 configured to receive a test consumable receptacle 221, 222, 223, 224, 225, 226 for either of an hsCTn test or a cCTn test. In some embodiments, a single test consumable receptacle 221 (or 222, 223, 224, 225, and/or 226) is configured to perform both an hsCTn test and cCTn test. In some embodiments, a first test consumable receptacle 221 is configured to perform an hsCTn test, and a second test consumable receptacle 222, 223, 224, 225, or 226 can be configured to perform a cCTn test. In some embodiments, the system 200 comprises a first port configured to receive a hsCTn test consumable receptacle, and a second port configured to receive a cCTn test consumable receptacle (e.g., the system can comprise about two or more ports 210, for example about two, three, four, five, six, seven, eight, nine, or ten ports, including ranges between any two of the listed values). The system can comprise a display 230 configured to display text and/or images 231, for example the results of a test. In some embodiments, the display 230 comprises a touch screen, which can further be used to calibrate, operate, and/or control the system. The system can comprise a detector 240 configured to detect the results of an hsCTn and/or cCTn test performed on a test consumable receptacle. The system can comprise a processor 250 in data communication with the detector 240, in which the processor 250 configured to process information detected by the detector 240, and to produce different signals indicative of different cardiac troponin test results, for either or both of the hsCTn and cCTn tests. In some embodiments, the system further comprises a dispenser 260 configured to dispense test consumable receptacles as described herein. In some embodiments, the system does not comprise a dispenser 260, and, for example, a user can obtain test consumable receptacles from kit, for example in a pouch, bag, canister, box, blister pack or the like as described herein. In some embodiments, the kit also contains instructions, such as print or electronic instructions, to us the test consumable receptacles of the kit in a method as described herein. In some embodiments, six test consumable receptacles (3 hsCTn test consumable receptacles and 3 cCTn test consumable receptacles) are provided together in a package, container, kit, or the like, so that a user can use these test consumable receptacles for a method as described herein. In some embodiments, the six test consumable receptacles are labeled to direct the user to perform a suitable sequence of CTn tests (hsCTn and cCTn) for the methods as described herein.

As illustrated by way of example in FIG. 6, a system 200 for determining AMI in accordance with some embodiments herein can comprise a processor 250. The processor 250 can be in data communication with a detector 240 configured to detect the results of a hsCTn or cCTn test from one or more test consumable receptacles 221, 222, 223, 224, 225, 226 positioned in at least one port 210. In some embodiments, a single test consumable receptacle is configured to perform both an hsCTn test and cCTn test. In some embodiments, a first test consumable receptacle is configured to perform an hsCTn test, and a second test consumable receptacle can be configured to perform a cCTn test. The system can display the results of the test(s) on a display 230 in data communication with the processor. In some embodiments, the system further comprises a user-alert or reporting function and/or structure, such as a visible notice (e.g., on a screen or with a flashing light or a protruding or flagging device), or a haptic notifier (e.g., a vibrating notifier positioned near a user's body), or user alert function and/or structure 235, such as an audio output device (e.g., a speaker or bell), a visible notice (e.g., on a screen or with a flashing light or a protruding or flagging device), or a haptic notifier (e.g., a vibrating notifier positioned near a user's body) in data communication with the port 210. The user-alert function and/or structures, such as the user alert function and/or structure 235, can be configured to produce “alerts” to notify a user (e.g. a health care provider such as a nurse or physician) of an event, for example one or more visible signals or chimes or bells or vibrations to notify the user that the next cardiac troponin test is to be performed, and/or to produce “alarms” to notify the user of an error. In some embodiments, the user alert function and/or structure 235 notifies the user of an error when a tag of the test consumable receptacle 221, 222, 223, 224, 225, 226 does not match the instant test type (hsCTn or cCTn), phase, and/or patient identity. In some embodiments, the user alert function and/or structure 235 is further in data communication with the processor 250 (via the port or directly). In some embodiments, the system comprises a single port 210 that accommodates both hsCTn and cCTn test consumable receptacles. The processor can adjust sensitivity and selectivity cutoffs as applicable so as to obtain the results of the hsCTn and cCTn test from the port. In some embodiments, the system 200 comprises a timer (for example as part of the processor 250, or spate from the processor), and the timer can track phases of a method as described herein. In some embodiments, an incongruity between the tag of a test consumable receptacle 221, 222, 223, 224, 225, 226 and the phase as indicated by the timer can indicate that an incorrect test consumable receptacle 221, 222, 223, 224, 225, or 226 has been positioned for reading by the port 210. As such, in response to the incongruity the user alert function and/or structure 235 can produce alarms as described herein to alert the user of an error. In some embodiments, the processor 250 adjusts sensitivity and selectivity cutoffs based on timing by the timer so that the results of a CTn test at a given phase of a method as described herein are read with a suitable level of sensitivity and selectivity.

In some embodiments, the user alert function and/or structure 235 can be configured to produce alarm sounds (such as bells, sirens, or the like), an alarm haptic notifier (e.g., a vibrating notifier positioned near a user's body), or an alarm visible notice (e.g., on a screen or with a flashing light or a protruding or flagging device, for example alight or the display 230) to signal an alarm, or the system can issue a combined audible, visible, and/or haptic alarm (such as a sound produced by the user alert function and/or structure 235 in conjunction with a flashing light or illuminated display 230) to notify the user of an error, for example if an incorrect type of test consumable receptacle 221, 222, 223, 224, 225, and/or 226 is positioned for detection by the detector (e.g. if a cCTn test consumable receptacle is positioned for detection when a hsCTn test is required), or if a test consumable receptacle 221, 222, 223, 224, 225, and/or 226 is positioned for detection by the detector. In some embodiments, the system further comprises a dispenser 260 configured to dispense test consumable receptacles 221, 222, 223, 224, 225, and/or 226. The dispenser 260 can be in data communication with the processor 250, for example via a wired connection and/or a wireless connection. The dispenser can dispense a type or types of test consumable receptacles (e.g. hsCTn and/or cCTn) in a manner and/or order and/or timing that correlate with a testing regimen for the subject. In some embodiments, the processor 250 can direct the timing and/or order and/or type of test consumable receptacles dispensed by the dispenser 260. In some embodiments, the system 200 can provide an alert, for example a visible, haptic, and/or audible alert by the user alert function and/or structure 235 as described herein (e.g., an audio output device such as a speaker or bell, a visible notice such as on a screen or with a flashing light or a protruding or flagging device, or a haptic notifier such as a vibrating notifier positioned near a user's body).chime or beep produced by an audio output device, a visible alert such as a flashing light or illuminated display 230, a haptic notification, and/or a combined audible, haptic, and/or visible alert) to notify a user that the test consumable receptacle 221, 222, 223, 224, 225, and/or 226 has been dispensed. In some embodiments, the test consumable receptacle 221, 222, 223, 224, 225, and/or 226 is provided in a container, for example a pouch, package, bag, canister, box, blister pack, or the link, which can be opened by a user. The test consumable receptacles can be labeled with letters, numbers, symbols, shapes, colors, or other suitable labels to direct the user to insert test consumable receptacles for hsCTn and cCTn tests in a sequence in accordance with the methods described herein.

In some embodiments, the dispenser 260 dispenses hsCTn and/or cCTn test consumable receptacles 222, 223. In some embodiments, the dispenser 260 is configured to hold at least about 2 test consumable receptacles, for example at least about 2, 3, 4, 5, 6, 7, 8, 9, 10, 15, 20, 25, 30, 35, 40, 45, 50, 60, 70, 80, 90, 100, 150, 200, 250, 300, 350, 400, 450, or 500 test consumable receptacles, including ranges between any two of the listed values, until they are ready to be dispensed. The systems of embodiments herein can be configured to detect the results of cardiac troponin tests and determine AMI in conjunction with methods as described herein, for example methods as shown in FIG. 3. Any embodiment of the system 200 can be implemented in any of the following form factors: a single handheld device, a handheld device in conjunction with a base unit, or a benchtop (or tabletop) system. As used herein a “benchtop” system need not be placed on a table or bench, and can also refer to a system that can suitably be placed freestanding on a surface in general, such as a benchtop, tabletop, shelf, or floor. As such, “benchtop” systems also encompass larger, free-standing systems that can be placed on the floor, for example as can be used by a central lab. It is contemplated that that large form factors, such as central lab system can contain a sufficient number of ports 210 to simultaneously accommodate one or more users and/or one or more, and detect and process the results of the test.

In some embodiments, the system 200 comprises a single integrated unit, such as a handheld unit form factor, for example a tablet, candy bar form factor, box, or device that also comprises the port 210 and display 230 in a single chassis. In some embodiments, the single integrated unit can comprise two or more ports, for example one port configured to receive a test consumable receptacle for a hsCTn test, and one port configured to receive a test consumable receptacle for a cCTn test. In some embodiments, the system 200 comprises two or more separate modules in which the port 210 and detector 240 are part of a separate module, such as a wand or reader that is in data communication with the display. In some embodiments, the data communication is wireless. In some embodiments, the data communication is wired. In some embodiments, the processor 250 is part of the same module as the port 210 and detector 240. In some embodiments, the processor 250 is separate from the module comprising the port 210 and detector 240. For example, the processor 250 can be part of the same module as the display 230. In some embodiments, the handheld unit can comprise a user alert function and/or structure 235 such as an audio output device (e.g., a speaker, bell, or the like, which can be configured to produce alert chimes or beeps), a visible notice (e.g., on a screen or with a flashing light or a protruding or flagging device), and/or a haptic notifier (e.g., a vibrating notifier positioned near a user's body), which can notify the user that a subsequent test is to be performed, and/or that a test result is available. Each of the user alert functions and/or structures that are described and/or illustrated anywhere in this specification can be used to notify a user of any step or process or action described or illustrated anywhere in this specification. The user alert function and/or structure 235 can further be configured to produce alarms, which can notify the user of an error, for example the placement of an incorrect type of test consumable receptacle at a particular phase (e.g. placement of an hsCTn test consumable receptacle for reading at the port 210 when a cCTn test consumable receptacle is needed, or signaling that a test consumable receptacle for the incorrect subject has been positioned for reading at the port 210), and/or at an incorrect time (e.g. placement of a test consumable receptacle positioned for reading at the port at a time when no test is required).

For example, in some embodiments, the system 200 comprises a handheld unit comprising the display 230 in data communication with the processor 250 and a base unit comprising at least one a detector 240 configured to detect the results of a hsCTn or cCTn test from one or more test consumable receptacles 221, 222, 223, 224, 225, 226 positioned in at least one port 210. The detector 240 can also be in data communication with the processor 250. The processor 250 can be housed in either the handheld unit or the base unit. The display 230 on the handheld unit can provide a user, such as a health care practitioner, with information 231 such as test results, and alerts, timers, and the like indicating the next test to be performed (hsCTn or cCTn). In some embodiments, the base unit can comprise a dispenser 260 as described herein. In some embodiments, the handheld unit can comprise a user alert function and/or structure 235, such as a visible notice (e.g., on a screen or with a flashing light or a protruding or flagging device), a haptic notifier (e.g., a vibrating notifier positioned near a user's body), and/or an audio output device (such as a speaker, bell, or the like), as described herein. The user alert function and/or structure 235 can be configured to provide the user with alerts (for example, noting an upcoming subsequent test) and/or alarms (for example, noting the placement of an incorrect test consumable receptacle and/or an incorrect timing. The user alert function and/or structure 235 can be in data communication with the processor 250, and can be configured to sound alerts and/or alarms as described herein. The display 230 can be in data communication with the processor 250, for example via a wireless connection is the processor 250 is housed in the base unit, or wired connection if the processor 250 is housed in the handheld unit,

In some embodiments, the system 200 can comprise a handheld unit and a base unit in which the handheld unit comprises a port 210 and detector 240, and the base unit need not comprise any port 210 or detector 240. In some embodiments, the base unit can comprise the dispenser 260. In some embodiments, the handheld unit comprises the processor 250. In some embodiments, the base unit comprises the processor 250.

In some embodiments, the system 200 comprises a benchtop or tabletop unit comprising the processor 250, the display 230 in data communication with the processor 250, and at least one a detector 240 configured to detect the results of a hsCTn or cCTn test from one or more test consumable receptacles 221, 222, 223, 224, 225, and/or 226 positioned in at least one port 210. In some embodiments, the system 200 can comprise two or more different ports 210, and/or at least one port 210 configured to contain two or more different test consumable receptacles 221, 222, 223, 224, 225, and/or 226. The detector 240 can also be in data communication with the processor 250. The benchtop or tabletop unit can further comprise a display 230 in data communication with the detector. The benchtop or tabletop unit can further comprise an user alert function and/or structure 235, such as a visible notice (e.g., on a screen or with a flashing light or a protruding or flagging device), a haptic notifier (e.g., a vibrating notifier positioned near a user's body), and/or an audio output device (such as a speaker, bell, or the like) in data communication with the processor, so as to produce alerts and alarms as described herein. The benchtop or tabletop unit can further comprise a dispenser 260 as described herein. The benchtop or tabletop unit can also comprise a user alert function and/or structure 235 in data communication with the processor 250 as described herein.

In some embodiments, the system can be “locked,” so that a test consumable receptacle 221, 222, 223, 224, 225, 226 will only be read and/or test results will only be processed after an appropriate identifier has been provided to the system so as to “unlock” the system 200, either actively by the user or passively by an action performed by the user that automatically unlocks the system 200. A locking and/or unlocking feature can be used to enable or prevent any step or process or action described or illustrated anywhere in this specification. In some embodiments, the processor 250 can be configured to lock and unlock the system 200 upon receipt of an appropriate identifier. The identifier can comprise a code (such as a pin or passcode) or biometric information or a key that is on or associated with any component of the system, such as the test consumable receptacle (such as a magnetic key or RFID key or a bar code or an electronic signal or a physical shape of an object or an electrical connection provided by an object, or any other suitable identifier), and can be associated with a particular patient in some embodiments. In some embodiments, the identifier is comprised by a tag of the test consumable receptacle as described herein. In some embodiments, the identifier can be linked with or can enable or prevent a particular test sequence (e.g. one or more types of tests, timing or ordering of tests, the next kind of test needed, and the like) associated with a particular subject by the processor 250. Unlocking the system 200 can permit the system 200 to confirm the identity of the user, and/or the identity of the subject, and/or the phase or ordering or timing of any stage of the testing, for example the phase of the method as described in FIG. 3. As such, the system can monitor and/or regulate the sequence and/or timing of tests that are to be performed, and/or can ascertain whether a test consumable receptacle 221, 222, 223, 224, 225, 226 inserted into a port 210 of the system 200 corresponds to the correct user and/or subject and/or testing procedure, and whether the test consumable receptacle corresponds to the a correct kind of test to be performed for the phase of the method as described in FIG. 3 (e.g. hsCTn versus cCTn).

In some embodiments, a “lock” can diminish user error, for example, by sounding, displaying, or sounding and displaying an error alarm if an incorrect test consumable receptacle is inserted at a particular phase, or if any test consumable is inserted when no test consumable receptacle is required, or if test a consumable receptacle for the incorrect patient is inserted. In some embodiments, the user unlocks the system by entering a password or PIN into the system 200. In some embodiments, a user unlocks the system by providing biometric information to the system 200, for example a fingerprint or retina scan. In some embodiments, the user unlocks the system using a physical key for example an RFID or magnetic keycard, a barcode, an encrypted flash memory device, or a traditional key for insertion into a lock. In some embodiments, the dispenser 260 only dispenses test consumable receptacles 222, 223 after being unlocked, so that the correct kind of test consumable receptacle (hsCTn, cCTn, or hsCTn at the same time as cCTn) is distributed. In some embodiments, the test consumable receptacles 222, 223, each comprise an identifier such as a barcode, and the processor 250 pairs a particular identifier with the test sequence of a particular patient, so that upon later detecting the results of a test from a particular test consumable receptacle, the system can also match the results with a particular patient and test phase, and confirm that the correct test is being performed at the correct phase of a particular test sequence (e.g., a process as shown in FIG. 3). In some embodiments, upon recognition of an identifier for a particular test consumable receptacle, 222, 223, the processor 250 can thus recognize the patient, and can “unlock” the system so as to implement a sequence of kinds of cardiac troponin tests and timing of cardiac troponin tests associated with a particular patient, and in conjunction with methods described herein (e.g., methods as shown in FIG. 3). In some embodiments, upon recognition of an identifier for an incorrect test consumable receptacle, 222, 223 (e.g., out of sequence, or incorrect patient), the processor 250 can signal the system to issue an alarm, for example via a user alert function and/or structure 235 as described herein (e.g., user alert function and/or structure such as a visible notice (e.g., on a screen such as the display 230 or with a flashing light or a protruding or flagging device), or a haptic notifier (e.g., a vibrating notifier positioned near a user's body).

In some embodiments, the port 210 comprises an opening configured to receive the test consumable receptacle 221, 222, 223, 224, 225, 226. In some embodiments, the port 210 is configured to align an inserted test consumable receptacle 221, 222, 223, 224, 225, 226 with the detector 240. In some embodiments, the port 210 comprises a window or adapter configured to directly or indirectly contact the test consumable receptacle 221, 222, 223, 224, 225, 226, but does not receive the test consumable receptacle 221, 222, 223, 224, 225, 226. For example, the matrix of a lateral flow test can be places against a window of the port 210. In some embodiments, the port 210 is convex, and docks with a concave portion of the test consumable receptacle 220, 221.

Test consumable receptacles 221, 222, 223, 224, 225, 226 in accordance with some embodiments herein can comprise any suitable container, cartridge, test tube, substrate, gel, matrix, column, or the like for performing an hsCTn and/or cCTn test. For example, a test consumable receptacle can comprise a test tube configured to hold a no-wash assay. For example, a test consumable receptacle can comprise a matrix for a lateral flow assay. For example, a test consumable receptacle can comprise a substrate for an ELISA assay. In some embodiments a single test consumable receptacle 221, 222, 223, 224, 225, or 226 is configured to perform only one of an hsCTn test or cCTn test. For example a first test consumable 221 is configured to perform only an hsCTn test, and a second test consumable receptacle 222, 223, 224, 225, and/or 226 is configured to perform only a cCTn test. In some embodiments, a single test consumable receptacle 221, 222, 223, 224, 225, or 226 is configured to perform both an hsCTn test and cCTn test, for example by comprising reagents for a hsCTn test at a first position or stripe, and comprising reagents for a cCTn test at a second position or stripe.

The detector 240 can comprise a suitable detector based on the particular hsCTn and/or cCTn test, for example an optical, electromagnetic, NMR, or radioactivity detector. For example, the detector 240 can comprise an optical detector for a no-wash assay comprising a FRET pair. For example, the detector 240 can comprise an optical detector configured to detect an amount of colorimetric label from an enzymatic reaction of an ELISA test. For example, the detector 240 can comprise an electromagnetic detector 240 configured to quantity an amount of metal-labeled antibody immobilized on one or more portions of a lateral flow assay. For example, the detector 240 can comprise a Raman spectrometer configured to detect an amount of SERs-tag labeled antibody immobilized on an ELISA substrate or lateral flow matrix.

The processor 250 can be configured to process and/or compare raw test data from the detector 240 to ascertain a test result. For example, the processor 250 can be configured to calculate an amount of cardiac troponin detected in a test (hsCTn or cCTn) on a test consumable receptacle 221, 222, 223, 224, 225, 226 based on raw signal from the detector 240. In some embodiments, the raw signal can be compared to a control. In some embodiments, the control comprises a consumable receptacle having a known quantity of cardiac troponin, and the raw signal is detected by the detector 240 for calibration purposes. In some embodiments, the control comprises an optically or electronically stored value. The signal produced by the processor can be indicative of the test result, for example whether the amount of cardiac troponin detected (or the change in amount of detected cardiac troponin) is above, below, or equal to a particular value. In some embodiments, the value comprises a predetermined cutoff. In some embodiments, the value comprises an amount that depends on another test (for example a baseline cCTn test) and/or a control that is actually run on the system 200. For example, the processor can produce a first signal if the test result (hsCTn or cCTn) is positive, and a second signal if the test result (hsCTn or cCTn) is not positive (e.g. observational, or negative). For example, the processor can produce a first signal if the test result (hsCTn or cCTn) is not negative (e.g. positive or observational), and a second signal if the test result (hsCTn or cCTn) is negative. For example, the processor can produce a first signal if the test result (hsCTn or cCTn) is positive, a second signal if the test result (hsCTn) is observational, and a third signal if the test result (hsCTn or cCTn) is negative. In some embodiments, the processor can tag the signal, for example to annotate the signal with the type of test performed (e.g. hsCTn or cCTn), information about the sample (e.g. identifying information about the subject and/or the period from which the sample was obtained from the subject), information about the test (e.g. time of test, location of test, raw values obtained, and/or statistical analysis). In some embodiments, the processor 250 is configured to produce a recommendation, based on particular test result. For example, the recommendation from the processor can represent an intermediate or ultimate outcome of FIG. 3. For example, the processor can recommend a second cCTn test 120 if the first hsCTn test 110 is positive 111. For example, the processor can rule-out AMI 163 if the third hsCTn test 130 is not positive 132. For example, the process can determine a presence of AMI if the second CCTN test 120 is positive 121. The display 230 can be configured to present the test results (hsCTn and/or cCTn), recommendation, and or data identified by tags from the signal produced by the processor 250.

In some embodiments, one or more existing systems for detecting results of cCTn and/or hsCTn can be used with suitable modifications. In some embodiments, one of the following systems can be configured, for example by reprogramming and/or attachment of suitable modules, to comprise the characteristics of systems described in any embodiments herein, for example those of FIGS. 4A-B and/or FIG. 6. Example systems that can be configured as such include a PATHFAST system (Mitsubishi Chemical Medicine), an ADVIA CENTAUR XP/CP system (Siemens AG), a STRATUS CS system (Siemens AG), an ARCHITEXT i2000 system (Abbott), a MINIBAIDASU system (Sysmex), a BITOROSU 3600 system (Ortho), a EKURUSHISU 2010 system (LOCHES), an AIA360 system (Tosoh corporation), an EBANETTO EV20 system (Tosoh Corp.), an ISTAT 300F-S system (Abbott), a TRIAGE system (Inverness(BioSite)), an AxSYM system (Abbott), an Access 2 system (Beckman Coulter), a UNICEL system (Beckman Coulter), an OPUS system (Siemens AG), a DIMENSION VISTA system (Siemens AG), a NANO-CHECK system (Nano-ditech), A DXPRESS READER system (Princeton Biomeditech Corp.), an AQT90 system (Radiometer), a RAMP READER system (Response Biomedical Corp.), an IMMULITE system (Siemens AG), a TECHICON system (Siemens AG), a SentiLAB system (Sentinal Diagnostics), an ACL ACUSTAR system (Instrument Laboratory), a Liaison system (DisSorin), an AIO! system (Innotrac), a LABGEO IB10 system (Samsung) an ABX PENTRA 400 system (Hiriba), an Miniquant D-Dimer System (Trinity Biotech), or a SIGNULEX system (Erenna). In some embodiments, any one of the listed systems can be suitably configured to possess one or more characteristics of a system as described in some embodiments herein, for example in FIGS. 4A-B and/or FIG. 6. In some embodiments, any method or step described herein can be performed on one of the listed systems or one of the suitably modified systems.

In some embodiments, a single test consumable receptacle 221, 222, 223, 224, 225, or 226 comprises a nanotechnology-scale receptacle, for example a nanoliter-scale test consumable receptacle (e.g., configured for testing volumes of sample on the scale of about 1 nanoliter of sample to about 1000 nanoliters of sample). The nanotechnology-scale receptacle can be configured for nanoliter-scale detection using at least one of chemiluminescence (for example, “flash” chemiluminescence), fluorescence (for example, via fluorescently-labeled antibody, a fluorescence enzyme immunoassay, and/or time-resolved fluorescence), electrochemical emission detection, immunochromato detection, or spectrophotometric detection. In some embodiments, the nanoliter-scale test consumable receptacle is configured for a hsCTn and/or cCTn test comprising at least one of a Chemiluminescence immunoassay (CLEIA), a Fluorescent antibody method (e.g., a sandwich method, such as a sandwich ELISA or sandwich no-wash assay), a fluorescent immunoassay (FIA), a fluorescence enzyme immunoassay (FEIA), a chemiluminescence immunoassay (CLEIA), “flash” chemiluminescence, electrochemical continuous emission immunoassay (ECLIA), a fluorescent antibody (immunity) law, a time-resolved fluorescence method, (e.g., a EUROPIUM assay), an immunochromato assay, a spectrophotometric assay, a immunonephelometry, or a sandwich immunoassay.

In some embodiments, a single test consumable receptacle 221, 222, 223, 224, 225, or 226 comprises a lab-on-a-chip test consumable receptacle. The lab-on-a-chip test consumable receptacle can comprise a portable form factor test consumable receptacle configured for detection of CTn (hsCTn and/or cCTn) using methods as described herein, for example immunoassays, at a milliliter-, microliter-, or nanoliter-scale volume. For example, the lab-on-a-chip test consumable receptacle can comprise an inlet for loading a sample as described herein, for example through direct contact with a patient blood, saliva, and/or urine sample, such by pricking a patient to obtain blood, or positioning the lab-on-a chip consumable receptacle in the path of urine or saliva. In some embodiments, the lab on-a-chip consumable receptacle comprises one or more fluidic channels, which can be configured to contain nanoliter-scale (e.g., about 1 nanoliter to about 1000 nanoliters), microliter-scale (e.g., about 1 microliter to about 1000 microliters), or milliliter-scale (e.g., about 1 milliliter to about 1000 milliliters) amounts of sample fluid. In some embodiments, the lab-on-a-chip test consumable receptacle is configured for test results to be received and analyzed on a smartphone, tablet, personal computer, or the like, and can be configured to be in data communication with the smartphone, tablet, personal computer, or the like, for example via wireless protocol, or via a wired protocol, such as reader port. As such, in some embodiments a lab-on-a-chip testy consumable receptacle can collect a nanoliter-scale, microliter-scale, or milliliter-scale sample directly from a patient, and the results of the hsCTn and/or cCTn immunoassay can be detected using a portable device such as a smartphone, table, or laptop-computer, or using a stationary form-factor computer such a desktop computer. The sample fluid can be contacted in the lab-on-a-chip sample receptacle with one or more hsCTn and/or cCTn immunoassay reagents as described herein, for example an antibody, so that an hsCTn and/or cCTn test as described herein can be performed. In some embodiments, the lab-on-a-chip test consumable receptacle comprises a sensor for detecting an amount of hsCTn and/or cCTn bound to the immunoassay reagents, for example an electrical, electromagnetic, or optical sensor. In some embodiments, the lab-on-a-chip test consumable receptacle is configured for detection of at least one of chemiluminescence (for example, “flash” chemiluminescence), fluorescence (for example, via fluorescently-labeled antibody, a fluorescence enzyme immunoassay, and/or time-resolved fluorescence), electrochemical emission detection, immunochromato detection, or spectrophotometric detection. In some embodiments, the lab-on-a-chip test consumable receptacle is configured for a hsCTn and/or cCTn test comprising at least one of a Chemiluminescence immunoassay (CLEIA), a Fluorescent antibody method (e.g., a sandwich method, such as a sandwich ELISA or sandwich no-wash assay), a fluorescent immunoassay (FIA), a fluorescence enzyme immunoassay (FEIA), a chemiluminescence immunoassay (CLEIA), “flash” chemiluminescence, electrochemical continuous emission immunoassay (ECLIA), a fluorescent antibody (immunity) law, a time-resolved fluorescence method, (e.g., a EUROPIUM assay), an immunochromato assay, a spectrophotometric assay, a immunonephelometry, or a sandwich immunoassay. In some embodiments a lab-on-a-chip test consumable receptacle comprises an hsCTn test consumable receptacle. In some embodiments a lab-on-a-chip test consumable receptacle comprises an cCTn test consumable receptacle. In some embodiments a lab-on-a-chip test consumable receptacle comprises an hsCTn test consumable receptacle and a cCTn test consumable receptacle, for example two labs-on-a-chip in a stack or in a tandem form factor. In some embodiments, a lab-on-a-chip form factor test receptacle has external dimensions of less than or equal to about 4 cm×less than or equal to about 3 cm, and a thickness of less than or equal to about 5 mm. In some embodiments, a lab-on-a-chip form factor test receptacle has external dimensions of less than or equal to about 3 cm×less than or equal to about 2 cm, and a thickness of less than or equal to about 3 mm.

### Kits

In some embodiments, kits are provided. The kit can comprise reagents for performing the methods herein. In some embodiments, the kit comprises hsCTn and cCTn tests (e.g. test consumable receptacles, plus one or more other items or reagents for performing the test) so that a system as described herein can be used to perform a method of determining AMI as described herein.

In some embodiments, the kit comprises a first hsCTn test comprising (i) an initial hsCTn test for use on an initial sample obtained from the subject at the time the subject presents with acute coronary symptoms. The kit can comprise a subsequent hsCTn test on a subsequent sample obtained from the subject at a one-hour phase after the subject presents with acute coronary symptoms, such that if the first hsCTn test is negative, acute myocardial infarction (AMI) is ruled out. The kit can comprise a second conventional cardiac troponin (cCTn) test for use on a second sample obtained at a three-hour phase after the subject presents with acute coronary symptoms, and a third cCTn test for use on a third sample obtained from the subject at a four-hour phase after the subject presents with acute coronary symptoms, either or both of which can be used if the first hsCTn test is not negative. It is noted that if the second cCTn test is or third cCTn test is positive, the subject can be determined to have AMI. The kit can comprise a fourth cCTn test for use on a fourth sample obtained from the subject at a six-hour phase after the subject presents with acute coronary symptoms, such that if the fourth cCTn test is negative, no AMI treatment is required for the subject, and such that if the fourth cCTn test is not negative, the subject remains a candidate for conventional 6-12 hour AMI observation.

In some embodiments, the kit comprises, consists of, or consists essentially of about six consumable receptacles (for example 3 hsCTn test consumable receptacles and 3 cCTn test consumable receptacles, or 4 hsCTn test consumable receptacles and 4 cCTn test consumable receptacles, or 4 hsCTn test consumable receptacles and 3 cCTn test consumable receptacles, or 3 hsCTn test consumable receptacles and 4 cCTn test consumable receptacles). In some embodiments, six test consumable receptacles (3 hsCTn test consumable receptacles and 3 cCTn test consumable receptacles) are provided together in a package, container, bag, or the like, so that a user can use these test consumable receptacles for a method as described herein. In some embodiments, each test consumable receptacle of the kit comprises a label, which can direct the user to use the test consumable receptacle in a sequence that is in accordance with methods as described herein (for example, use of a suitable hsCTn or cCTn test consumable receptacle at a suitable phase as described herein). In some embodiments, each test consumable receptacle of the kit comprises a tag, so as to identify the test consumable as belonging to that particular kit. Accordingly, if a kit is used for a particular patient, the tag can also associate the test (and test results) of the various phases of the method with a particular patient.

In some embodiments, a method of instructing a user to determine acute myocardial infarction (AMI) in a subject that presents with acute coronary symptoms is provided. The method can comprise providing a user with a kit. The kit can comprise a first hsCTn test comprising (i) an initial hsCTn test consumable receptacle, and (ii) a subsequent hsCTn test consumable receptacle. The kit can comprise a second cCTn test consumable receptacle. The kit can comprise a third cCTn test consumable receptacle. Each test can be performed by contacting a sample with the test consumable receptacle(s) of that particular test. The method can comprise instructing the user to perform the initial hsCTn test on an initial sample obtained from the subject at the time the subject presents with acute coronary symptoms, and to perform the subsequent hsCTn test on a subsequent sample obtained from the subject no more than one hour after the subject presents with acute coronary symptoms. It can be noted that if the first hsCTn test is negative, acute myocardial infarction (AMI) is ruled out. The method can further comprise instructing the user that if AMI is not ruled out by the subsequent hsCTn test, to perform at least one of: (i) the second cCTn test on a second sample obtained from the subject at a three-hour phase after the subject presents with acute coronary symptoms; or (ii) the third cCTn test on a third sample obtained from the subject at a four-hour phase after the subject presents with acute coronary symptoms. The method can comprise instructing the user to recommend treating the subject for AMI if the second cCTn test or third cCTn test is positive. In some embodiments, the kit can further comprise a third hsCTn test. The method can comprise instructing the user to perform the third hsCTn test if the first hsCTn test is observational or if the second cCTn test is not positive. The method can further comprise instructing the user to perform the third cCTn test if the third hsCTn test is positive. In some embodiments, the method can further comprise instructing the user to recommend conventional 6-12 hour AMI observation if the third cCTn test is not negative. In some embodiments, the user can comprise a medical care provider, a diagnostic laboratory, or the like. In some embodiments, the hsCTn and cCTn test consumable receptacles of a particular kit comprise identifiers (such as one or more barcodes and/or RFID chips and/or physical shapes of the consumable receptacle and/or electronic signals and/or electronic connections, etc.), so that test consumable receptacles can be tracked, associated with a particular subject, and/or associated with a particular phase of a method, for example a method as shown in FIG. 3. In some embodiments, the test consumable receptacles are configured (e.g., by way of one or more incompatible physical interfaces or connections and/or by way of one or more improper identifiers, etc.) so that test consumable receptacles from different kits cannot be used with each other. It is noted that such a feature reduces the risk that incorrect patient samples with be run at particular phases of testing by identifying all of the test consumable receptacles that go together.

In some embodiments, a method as described above is performed using a mCTn test instead of or in addition to a hsCTn test, in conjunction with a cCTn that has a CV at the about 99th percentile value that is greater than or equal to about 20%.

Any feature, structure, method, step, or material described and/or illustrated in any embodiment of this specification can be used with or instead of any feature, structure, method, step, or material described and/or illustrated in any other embodiment of this specification. There is no required or indispensable or essential combination of features.

### Additional Embodiments

Major advances have recently been achieved by the development of more sensitive cardiac troponin assays. High-sensitivity cardiac troponin assays, which allow measurement of even lower concentrations of cardiac troponin with high precision, have been shown to largely overcome the sensitivity deficit of conventional cardiac troponin assays within the first hours of presentation in the diagnosis of acute MI.

In a multicentre study (Reichlin et. al, “Prospective validation of a 1-hour algorithm to rule-out and rule-in acute myocardial infarction using a high-sensitivity cardiac troponin T assay.: Apr. 13, 2015. CMAJ 2015. DOI:10.1503/cmaj.141349) a high-sensitivity cardiac troponin T 1-hour algorithm was shown to allow accurate rule-out and rule-in of acute MI within 1 hour in up to 75% of patients. This algorithm is based on 2 concepts: (1) High-sensitivity cardiac troponin T being interpreted as a quantitative variable where the proportion of patients who have acute MI increases with increasing concentrations of cardiac troponin T; and (2) Early absolute changes in the concentrations (detected as high-sensitivity cardiac troponin T) within 1 hour provide incremental diagnostic information when added to baseline levels, with the combination acting as a reliable surrogate for late concentrations at 3 or 6 hours.

In accordance with some embodiments herein, a 1-hour algorithm can rule-out and rule-in acute myocardial infarction using a high-sensitivity cardiac troponin T assay. The algorithm can incorporate both baseline high-sensitivity cardiac troponin T levels and absolute changes in the levels within the first hour. The results from the multicentre study (Reichlin et. al, CMAJ 2015. DOI:10.1503/cmaj.141349) for the indicated one-hour algorithm provide a negative predictive value for acute MI in the rule-out zone defined only by high-sensitivity cardiac troponin T levels at presentation and the absolute change within 1 hour was 99.9%. Overall, as shown in Example 1, 59.5% of all patients could be assigned to the rule-out category.

The positive predictive value for acute MI in the rule-in zone was 78.2% (see Example 1). Many of the patients in the rule-in zone with a diagnosis other than acute MI did have conditions that usually still require coronary angiography for accurate diagnosis, including Takotsubo cardiomyopathy, myocarditis and unstable angina

The 1-hour algorithm overall assigned 75.9% of patients a definite process (either rule-out or rule-in)(see Example 1). Thereby, the high-sensitivity cardiac troponin T 1-hour algorithm was even more effective in the early triage of patients with acute chest pain

Cumulative 30-day mortality was 0.0% in patients assigned the rule-out zone, further documenting the safety of this approach and the suitability of many of these patients for early discharge

The clinical application of the high sensitivity cardiac troponin T 1-hour algorithm will profoundly affect the management of about 75% of patients, it will not affect or will only marginally affect the management of the about 25% of patients assigned the observational zone

The algorithm can potentially reduce the time to discharge from the emergency department by about 500%.

Methods, systems, and kits in accordance with some embodiments herein incorporate aspects of the 1 hour algorithm of high sensitive troponin in combination with conventional troponin test at various time intervals to accurately rule in and rule out patients with Acute MI with-in 6 hours of ED presentation.

Some examples of methods in accordance with some embodiments herein are divided into 5 phases with phase 1 at ED presentation, phase 2 at about 1 hour after ED presentation, phase 3 at about 3 hours after ED presentation, phase 4 at about 4 hours from ED presentation and phase 5 at about 6 hours from ED presentation. Some examples of various tests that can be carried out at different phases are as follows:

Phase 1 and phase 2 is the application of “1-hour algorithm to rule-out and rule-in acute myocardial infarction using a high-sensitivity cardiac troponin T assay.”

In Phase 3, conventional cardiac troponin test is performed on the Rule-In patients from 1-hour algorithm (phase 2) and patients are either ruled in and the remaining are sent to observational group. The major objective of this test is to rule out the false positive cases from phase 2 ruled in patients.

The phase 4 is divided into 2 steps: Step 1: high sensitive cardiac troponin test is carried on the observational group of patients from phase 2 and phase 3 and further classified into rule in and observational group, and Step 2: Conventional cardiac troponin test is carried out on the ruled in patients from step 1 so as to eliminate false positives.

Without being limited by any theory, phase 4 can identify the AMI patients from observational zone so that early intervention can be administered. Clinical data provides substantial evidence that early intervention provided to AMI patients with-in 4 hours of ED presentation has major impact on mortality and morbidity.

In phase 5, the conventional cardiac troponin test is done on the observational group of patients from phase 4 to rule out large amount of healthy patients and send the remaining patients to observational group who go through a conventional 6-12 cardiac care pathway.

### Example 1

cCTn and hsCTn results for 1000 candidate subjects who presented with acute coronary symptoms, but did not exhibit ST-elevation on ECG were assessed using methods in accordance with embodiments herein. These subjects underwent conventional AMI observation (and where applicable) conventional AMI treatment, and a meta-analysis was performed on iterative hsCTn and cCTn test results for samples obtained at various timepoints after the subject presented with acute coronary symptoms. According to conventional criteria, of 1000 candidate patients who presented with acute coronary symptoms, 176 had AMI, and 824 were “healthy”. The disease prevalence of AMI in this population was 17.6 percent.

The inputs for the analysis are summarized in Tables 1.1, 1.3, 1.4, 1.5, 1.6, and 1.7 below. Shown are sensitivity (SE), specificity (SP), positive predictive value (PPV), and negative predictive value (NPV). It is noted that optional Table 1.2 is provided for reference, but the calculations of Table 1.2 were not used in the methods of this Example.

TABLE 1.1
Input for Strategy 1 Stage 1 Results (Based on 1 hour algorithm of hsCTnT)

| | | PPV | | | NPV | | |
|---|---|---|---|---|---|---|---|
| Category of Patients | % | Low | High | 95% CI | Low | High | 95% CI |
| Rule In | 16.40% | 72.10% | 83.60% | 78.20% | | | |
| Rule Out | 59.50% | | | | 99.30% | 100% | 99.90% |
| Observational | 24.10% | | | | | | |

TABLE 1.2
At ED Presentation (optional)

| | cCTnI | | | hsCTnT | | |
|---|---|---|---|---|---|---|
| | Low | High | 95% CI | Low | High | 95% CI |
| SE | 0.57 | 0.67 | 0.65 | 0.593 | 0.931 | 0.8 |
| SP | 0.92 | 0.97 | 0.95 | 0.659 | 0.914 | 0.81 |
| PPV | | | | 50.90% | 87% | 71.48% |
| NPV | | | | 72.60% | 72.60% | 87.10% |

TABLE 1.3
Samples at three-hour phase after presentation with acute
coronary symptoms

| | cCTnI | | | hsCTnT | | |
|---|---|---|---|---|---|---|
| | Low | High | 95% CI | Low | High | 95% CI |
| SE | 0.55 | 0.84 | 0.71 | 0.651 | 0.955 | 0.846 |
| SP | 0.94 | 0.98 | 0.97 | 0.692 | 0.923 | 0.83 |
| PPV | | | | 53.70% | 88% | 73.35% |
| NPV | | | | 77.90% | 97.40% | 90.69% |

TABLE 1.4
Samples at four-hour phase after presentation with acute
coronary symptoms

| | cCTnI | | | hsCTnT | | |
|---|---|---|---|---|---|---|
| | Low | High | 95% CI | Low | High | 95% CI |
| SE | 0.55 | 0.84 | 0.71 | 0.803 | 0.994 | 0.962 |
| SP | 0.94 | 0.98 | 0.97 | 0.692 | 0.923 | 0.83 |
| PPV | | | | 57.40% | 89% | 75.78% |
| NPV | | | | 86.80% | 99.60% | 97.53% |

TABLE 1.5
Samples at six-hour phase after presentation
with acute coronary symptoms

| | cCTnI | | |
|---|---|---|---|
| | Low | High | 95% CI |
| SE | 0.817 | 0.908 | 0.868 |
| SP | 0.9 | 0.941 | 0.922 |
| PPV | 73.80% | 84.10% | 79.30% |
| NPV | 93.40% | 96.80% | 95.30% |

TABLE 1.6
Samples at twelve-hour phase after presentation
with acute coronary symptoms

| | cCTnI | | |
|---|---|---|---|
| | Low | High | 95% CI |
| SE | 0.837 | 0.916 | 0.88 |
| SP | 0.9 | 0.938 | 0.92 |
| PPV | 74.10% | 86.10% | 78.70% |
| NPV | 85.50% | 90.30% | 95.90% |

TABLE 1.7
AMI Prevalence among patients presented at ED with chest pain

| | | |
|---|---|---|
| Low | 12.50% | 125 |
| High | 25% | 250 |
| Average | | 188 |

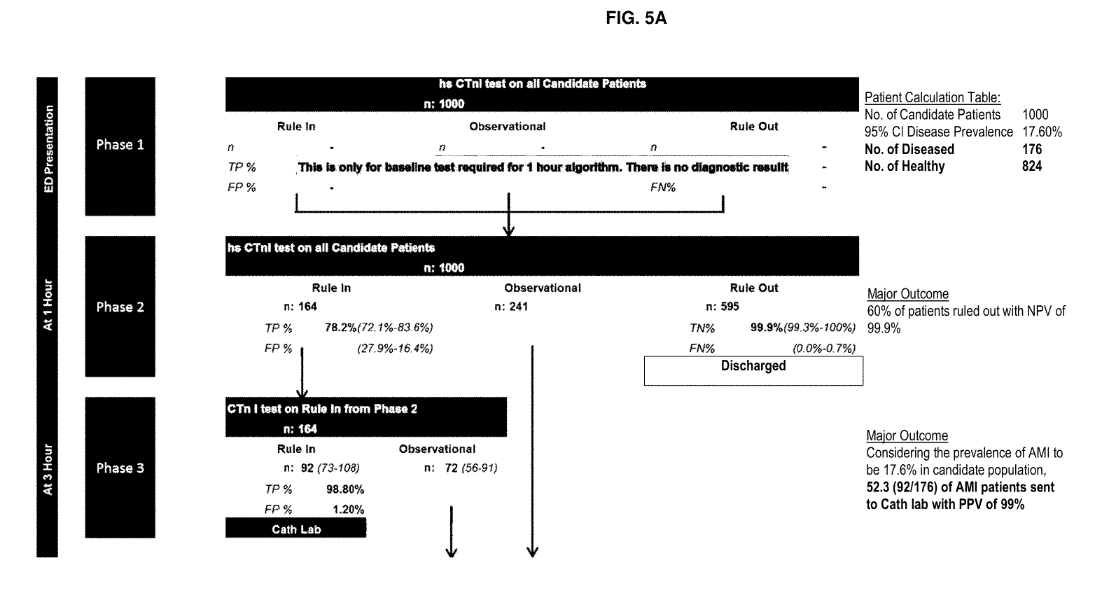

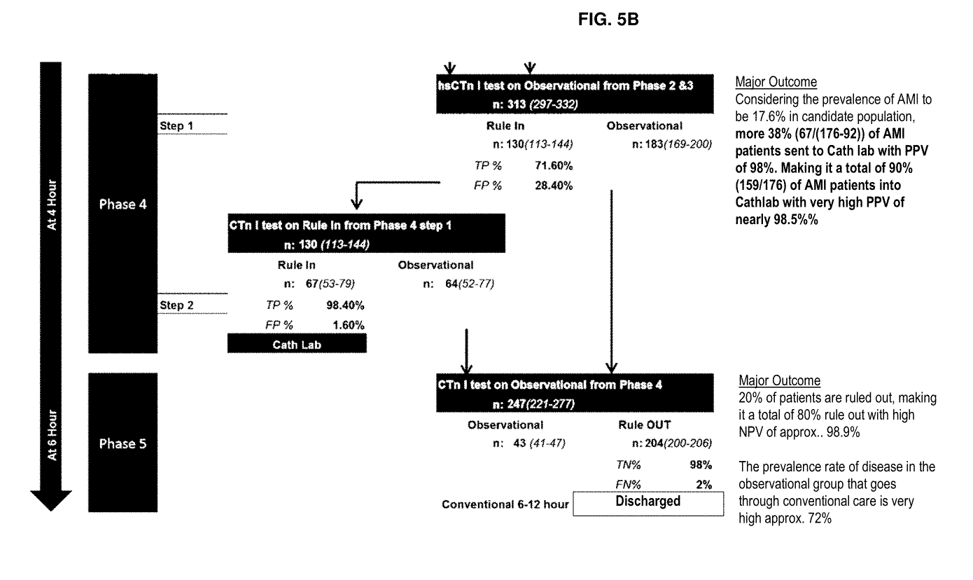

The cCTn and hsCTn test results were applied to the method of the flow diagram shown in FIG. 3. As shown in FIG. 5A, the first hsCTn test was performed, comprising an initial hsCTn test on a sample taken at the time the subjects presented with acute coronary symptoms (see FIG. 5A at “Phase 1”), and a subsequent hsCTn test on a sample obtained from each subject at the one-hour phase after the subject presented with acute coronary symptoms (see FIG. 5A at “Phase 2”). For the first hsCTn test, 595 subjects were negative (a true negative rate of 99.9%), 241 subjects were observational, and 164 were positive (a true positive rate of 78.3%). Thus, 60% of the patients were ruled-out for AMI after the first hsCTn test (with a true negative rate of 99.9%).

For the subjects who were positive for the first hsCTn test, a second cCTn test was performed on a sample obtained from each subject at the three-hour phase after the subject presented with acute coronary symptoms (see FIG. 5A at “Phase 3”). 92 of the 164 subjects tested positive in the second cCTn test, and were determined to have AMI (a true positive rate of 98.8%). 72 of the 164 subjects tested as “not positive” (e.g. “observational”) in the second cCTn test (see FIG. 5A at “Phase 3”). Thus, 52.3% of the patients calculated to be true positives for AMI were determined as positive for AMI by methods in accordance with some embodiments herein, and could be recommended for AMI treatment after the second cCTn test, which was performed on the sample obtained at the three-hour phase after the subject presented with acute coronary symptoms.

For the 72 subjects tested as “not positive” in the second cCTn test and the 241 subjects tested as “observational” in the first hsCTn test, a third hsCTn test was performed on a sample obtained from each subject at the four-hour phase after the subject presented with acute coronary symptoms (see FIG. 5B at “Phase 4, Step 1”). Of the 313 subjects tested in the third hsCTn test, 130 were tested as positive (a true positive rate of 71.6%, and the remaining 183 were tested as “observational”. For the 130 subjects who tested as “positive” in the third hsCTn test, a third cCTn test was performed on the sample obtained from each subject at the four-hour phase after the subject presented with acute coronary symptoms (see FIG. 5B at “Phase 4, Step 2”). In the third cCTn test, 67 of the subjects tested “positive”, and were determined to have AMI (a true positive rate of 98.4%). In the third cCTn test, 64 of the subjects tested as “not positive” (e.g. “observational”). Thus, 38% of the patients calculated to be true positives for AMI were determined as positive for AMI, and could be recommended for AMI treatment after the third cCTn test, which was performed on the sample obtained at the four-hour phase after the subject presented with acute coronary symptoms. In the aggregate, after the third cCTn test, 84% of the total patients calculated to be true positives (157/188) had been determined to have AMI, and could be recommended for AMI treatment following the cCTn testing of the third sample, obtained at the four-hour phase after the subject presented with acute coronary symptoms.

For the 64 subjects tested as “not positive” in the third cCTn test, and the 183 subjects tested as “observational” in the third hsCTn test, a fourth cCTn test was performed on the sample obtained from each subject at the six-hour phase after the subject presented with acute coronary symptoms (see FIG. 5B at “Phase 5”). For the fourth cCTn test, 204 of the 247 subjects were tested as negative, and determined to not have AMI (a true negative rate of 98%). For the fourth cCTn test, the remaining 43 subjects (4.3% of the total subjects assessed) were determined to possibly have AMI, and were recommended for conventional 6-12 hour AMI observation. Following the fourth cCTn test, 20% of the patients were ruled out for AMI, making for a total of 80% rule out with a high total negative predictive value of 98.9%. Moreover, over 4.3% (43/1000) of the population had to undergo the “conventional” AMI observation for 6-12 hours after presenting with symptoms, and the prevalence of AMI in this group was very high (72%), considerably higher than the prevalence of AMI in the total population (17.6%).

Thus, of 1000 subjects determined to have AMI via methods in accordance with some embodiments herein, and treated for AMI via methods in accordance with some embodiments herein, 95.7% were determined to be positive or negative for AMI through the testing of samples obtained at or before the 6-hour phase after the subjects presented with acute coronary symptoms (and NSTEMI ECG). Many of the subjects were determined to be negative for AMI at a relatively early testing round, and did not need to undergo further testing, or invasive (and potentially risky) AMI treatment that was very likely unnecessary. Additionally, many of the subjects were determined to have AMI at or prior to the testing of samples obtained at the 6-hour phase after the subjects presented with acute coronary symptoms, and thus received AMI treatment earlier than a patient being tested and observed via conventional approaches.

The results of the analysis comprising hsCTn and cCTn tests in accordance with some embodiments herein are summarized in Tables 2.1, 2.2, 2.3, 2.4, 2.5, and 3 below. Table 3 below summarizes the consolidated results.

TABLE 2.1
Assessment comprising hsCTn and cCTn

| | | Category | | | | Prevelance | | |
|---|---|---|---|---|---|---|---|---|
| Time | Test | of | # of | Disease | Healthy | of | Adjusted | Adjusted |
| Point | Done | Patient | Patients | population | Population | Diseases | PPV | NPV |
| ED | hsCTnT | ALL | 1000 | — | — | 19% | — | — |
| Presentation | | | | | | | | |
| 1 Hour | hsCTnT | ALL | 1000 | — | — | 19% | — | — |
| Phase | | | | | | | | |
| 3 Hour | CTnI | Rule IN | 164 | 128 | 36 | 78% | 99% | — |
| Phase | | from 1 | | | | | | |
| | | hour | | | | | | |
| 4 Hour | hsCTnT | Observ. | 241 | | | | | |
| Phase | | From 1 | | | | | | |
| | | hour | | | | | | |
| | hsCTnT | Observ. | 72 | | | | | |
| | | From 3 | | | | | | |
| | | hour | | | | | | |
| | | Total | 313 | 96 | 216 | 31% | 72% | — |
| | CTnI | Rule IN | 130 | 91 | 37 | 72% | 98% | — |
| | | from 3 | | | | | | |
| | | hour | | | | | | |
| 6 Hour | CTnI | Observ. | 247 | 31 | 216 | 12% | 61% | 98% |
| Phase | | from 4 | | | | | | |
| | | hour | | | | | | |

TABLE 2.2
Assessment comprising hsCTn and cCTn

| | | Category | Rule IN | | | Rule OUT | | |
|---|---|---|---|---|---|---|---|---|
| Time | Test | of | | | 95% | | | 95% |
| Point | Done | Patient | Low | High | CI | Low | High | CI |
| ED | hsCTnT | ALL | — | — | — | — | — | — |
| Presen- | | | | | | | | |
| tation | | | | | | | | |
| 1 Hour | hsCTnT | ALL | — | — | 164 | — | — | 595 |
| Phase | | | | | | | | |
| 3 Hour | CTnI | Rule IN | 73 | 108 | 92 | — | — | — |
| Phase | | from 1 | | | | | | |
| | | hour | | | | | | |
| 4 Hour | hsCTnT | Observ. | | | | | | |
| Phase | | From 1 | | | | | | |
| | | hour | | | | | | |
| | hsCTnT | Observ. | | | | | | |
| | | From 3 | | | | | | |
| | | hour | | | | | | |
| | | Total | 144 | 113 | 130 | — | — | — |
| | CTnI | Rule IN | 53 | 79 | 67 | — | — | — |
| | | from 3 | | | | | | |
| | | hour | | | | | | |
| 6 Hour | CTnI | Observ. | | | | 200 | 206 | 204 |
| Phase | | from 4 | | | | | | |
| | | hour | | | | | | |

TABLE 2.3
Assessment comprising hsCTn and cCTn

| | | Cate-gory | Observational | | |
|---|---|---|---|---|---|
| Time Point | Test Done | of Patient | Low | High | 95% CI |
| ED | hsCTnT | ALL | — | — | — |
| Presentation | | | | | |
| 1 Hour Phase | hsCTnT | ALL | — | — | 241 |
| 3 Hour Phase | CTnI | Rule IN | 91 | 56 | 72 |
| | | from 1 hour | | | |
| 4 Hour Phase | hsCTnT | Observ. | | | |
| | | From 1 hour | | | |
| | hsCTnT | Observ. | | | |
| | | From 3 hour | | | |
| | | Total | 169 | 200 | 183 |
| | CTnI | Rule IN | 77 | 52 | 64 |
| | | from 3 hour | | | |
| 6 Hour Phase | CTnI | Observ. | 47 | 41 | 43 |
| | | from 4 hour | | | |

TABLE 2.4
Assessment comprising hsCTn and cCTn

| | | Category | True Positives | | | False Positives | | |
|---|---|---|---|---|---|---|---|---|
| Time | Test | of | | | 95% | | | 95% |
| Point | Done | Patient | Low | High | CI | Low | High | CI |
| ED | hsCTnT | ALL | — | — | — | — | — | — |
| Presen- | | | | | | | | |
| tation | | | | | | | | |
| 1 Hour | hsCTnT | ALL | 118 | 137 | 128 | 46 | 27 | 36 |
| Phase | | | | | | | | |
| 3 Hour | CTnI | Rule IN | 72 | 107 | 91 | 1 | 1 | 1 |
| Phase | | from 1 | | | | | | |
| | | hour | | | | | | |
| 4 Hour | hsCTnT | Observ. | | | | | | |
| Phase | | From 1 | | | | | | |
| | | hour | | | | | | |
| | hsCTnT | Observ. | | | | | | |
| | | From 3 | | | | | | |
| | | hour | | | | | | |
| | | Total | 103 | 81 | 93 | 41 | 32 | 37 |
| | CTnI | Rule IN | 52 | 77 | 66 | 1 | 1 | 1 |
| | | from 3 | | | | | | |
| | | hour | | | | | | |
| 6 Hour | CTnI | Observ. | | | | | | |
| Phase | | from 4 | | | | | | |
| | | hour | | | | | | |

TABLE 2.5
Assessment comprising hsCTn and cC7Tn

| | | Category | True Negatives | | | False Negatives | | |
|---|---|---|---|---|---|---|---|---|
| Time | Test | of | | | 95% | | | 95% |
| Point | Done | Patient | Low | High | CI | Low | High | CI |
| ED | hsCTnT | ALL | — | — | — | — | — | — |
| Presen- | | | | | | | | |
| tation | | | | | | | | |
| 1 Hour | hsCTnT | ALL | 591 | 595 | 594 | 4 | 0 | 1 |
| Phase | | | | | | | | |
| 3 Hour | CTnI | Rule IN | — | — | — | — | — | — |
| Phase | | from 1 | | | | | | |
| | | hour | | | | | | |
| 4 Hour | hsCTnT | Observ. | | | | | | |
| Phase | | From 1 | | | | | | |
| | | hour | | | | | | |
| | hsCTnT | Observ. | | | | | | |
| | | From 3 | | | | | | |
| | | hour | | | | | | |
| | | Total | — | — | — | — | — | — |
| | CTnI | Rule IN | — | — | — | — | — | — |
| | | from 3 | | | | | | |
| | | hour | | | | | | |
| 6 Hour | CTnI | Observ. | 196 | 202 | 200 | 4 | 4 | 4 |
| Phase | | from 4 | | | | | | |
| | | hour | | | | | | |

TABLE 3
Consolidated result

| Rule IN | | | True Positives | | | False Positives | | |
|---|---|---|---|---|---|---|---|---|
| Low | High | 95% CI | Low | High | 95% CI | Low | High | 95% CI |
| 126 | 187 | 159 | 124 | 185 | 157 | 2 | 3 | 2 |
| Rule Out | | | True Negatives | | | False Negatives | | |
| Low | High | 95% CI | Low | High | 95% CI | Low | High | 95% CI |
| — | — | 795 | 787 | 797 | 794 | 8 | 4 | 5 |
| Observational | | | Diseased | | | Healthy | | |
| Low | High | 95% CI | Low | High | 95% CI | Low | High | 95% CI |
| 47 | 41 | 43 | 55 | 3 | 31 | −9 | 38 | 13 |

1. A method of treating a subject that presents with acute coronary symptoms, the method comprising:
performing a first high-sensitivity cardiac troponin (hsCTn) test comprising:
an initial hsCTn test on an initial sample obtained from the subject at the time the subject presents with acute coronary symptoms; and
a subsequent hsCTn test on a subsequent sample obtained from the subject at a one-hour phase after the subject presents with acute coronary symptoms,
wherein if the first hsCTn test is negative, acute myocardial infarction (AMI) is ruled out;
if the first hsCTn test is not negative, performing at least one of:
a second conventional cardiac troponin (cCTn) test on a second sample obtained at a three-hour phase after the subject presents with acute coronary symptoms; or
a third cCTn test on a third sample obtained from the subject at a four-hour phase after the subject presents with acute coronary symptoms; and
if the second cCTn test or third cCTn test is positive, treating the subject for AMI.

2. The method of claim 1, wherein if the first hsCTn test is positive, the second cCTn test is performed on the second sample.

3. The method of any one of claims 1-2, wherein if the first hsCTn test-is observational or if the second cCTn test is observational, the method further comprises:
performing the third hsCTn test on the third sample,
wherein if the third hsCTn test is positive, a third cCTn test is performed on the third sample or on another sample obtained from the subject at the four-hour phase after the subject presents with acute coronary symptoms, and
wherein if the third hsCTn test is not positive, the third cCTn test is not required.

4. The method of claim 3, wherein if the third hsCTn test is not positive, or if the third cCTn test is not positive, the method further comprises:
performing a fourth cCTn test on a fourth sample obtained from the subject at a six-hour phase after the subject presents with acute coronary symptoms,
wherein if the fourth cCTn test is negative, no AMI treatment is required for the subject, and
wherein if the fourth cCTn test is not negative, the subject remains a candidate for conventional 6-12 hour AMI observation.

5. A method of determining acute myocardial infarction (AMI) in a subject who presents with acute coronary symptoms, the method comprising:
performing a first high-sensitivity cardiac troponin (hsCTn) test comprising:
receiving an initial sample obtained from the subject at the time the subject presents with acute coronary symptoms and performing an initial hsCTn test on the initial sample; and
receiving a subsequent sample obtained from the subject at a one-hour phase after the subject presents with acute coronary symptoms and performing a subsequent hsCTn test on the subsequent sample,
wherein if the first hsCTn test is negative, determining an absence of AMI; and
if the first hsCTn test is not negative:
receiving at least one of:
a second sample obtained from the subject at a four-hour phase after the subject presents with acute coronary symptoms; or
a third sample obtained from the subject from the subject at a four-hour phase after the subject presents with acute coronary symptoms; and
performing at least one of:
a second conventional cardiac troponin (cCTn) test on the second sample; or
a third cCTn test on the third sample, wherein if the second cCTn test or third cCTn test is positive, the subject is determined to have AMI.

6. The method of claim 5, wherein if the first hsCTn test is positive, the second cCTn test is performed on the second sample.

7. The method of any one of claims 5-6, wherein if the first hsCTn test is observational or if the second cCTn test is observational, the method further comprises:
performing the third hsCTn test on the third sample,
wherein if the third hsCTn test is positive, a third cCTn test is performed on the third sample or on another sample obtained from the subject at the four-hour phase after the subject presents with acute coronary symptoms, and
wherein if the third hsCTn test is not positive, the third cCTn test is not required.

8. The method of claim 7, wherein if the third hsCTn test is not positive, or if the third cCTn test is not positive, the method further comprises:
performing a fourth cCTn test on a fourth sample obtained from the subject at a six-hour phase after the subject presents with acute coronary symptoms,
wherein if the fourth cCTn test is negative, no AMI treatment is required for the subject, and
wherein if the fourth cCTn test is not negative, the subject remains a candidate for conventional 6-12 hour AMI observation.

9. A system for determining acute myocardial infarction (AMI), the system comprising:
a detector configured to detect a level of troponin if the detector receives a test on a sample from a subject in a subject that presents with acute coronary symptoms, wherein the detector is configured to detect troponin levels from a high-sensitivity cardiac troponin (hsCTn) test, and detect troponin levels from a conventional cardiac troponin (cCTn) test;
a processor configured to provide a determination of a presence or absence of AMI based on troponin levels detected by the detector,
wherein the processor signals that the processor has determined the absence of AMI if the processor determines the detector to detect a negative result of a first hsCTn test comprising:
an initial hsCTn test on an initial sample obtained from the subject at a time the subject presents with acute coronary symptoms; and
a subsequent hsCTn test on a subsequent sample obtained from the subject at a one-hour phase after the subject presents with acute coronary symptoms, wherein if the first hsCTn test is negative, AMI is ruled out, and
wherein the processor signals a proposal for at least one of the following if the processor determines the detector to detect a non-negative result of the first hsCTn test:
a second cCTn test on a second sample obtained from the subject at a three-hour phase after the subject presents with acute coronary symptoms; or
a third cCTn test on a third sample obtained from the subject at a four-hour phase after the subject presents with acute coronary symptoms; and
the processor signals that the processor had determined a presence of AMI if the processor determines the detector to detect a positive for the second or third cCTn test.

10. The system of claim 9, wherein if the processor determines a positive result for the first hsCTn test, the system is configured to perform the second cCTn test is performed on the second sample.

11. The system of any one of claims 9-10, wherein if the first hsCTn test is observational or if the second cCTn test is observational, the system is further configured to:
detect the result of the third hsCTn test on the third sample,
wherein if the third hsCTn test is positive, the processor is configured to send a signal to prompt a third cCTn test to be performed on the third sample or on another sample obtained from the subject at the four-hour phase after the subject presents with acute coronary symptoms,
wherein the system is configured to detect the results of the third cCTn test if it is performed, and
wherein if the third hsCTn test is not positive, the third cCTn test is not required.

12. The system of claim 11, wherein if the third hsCTn test is not positive, or if the third cCTn test is not positive, the system is further configured to:
detect the results of a fourth cCTn test on a fourth sample obtained from the subject at a six-hour phase after the subject presents with acute coronary symptoms,
wherein if the fourth cCTn test is negative, no AMI treatment is required for the subject, and
wherein if the fourth cCTn test is not negative, the subject remains a candidate for conventional 6-12 hour AMI observation.

13. The system of claim 9, further comprising a port configured to receive a test consumable receptacle for an hsCTn test, and to receive a test consumable receptacle for a cCTn test.

14. The system of claim 9, further comprising a first port configured to receive a test consumable receptacle for an hsCTn test, and a second port configured to receive a test consumable receptacle for a cCTn test.

15. The system of claim 13 or 14, wherein the port comprises a reader for a tag of a test consumable receptacle, wherein the tag identifies an association of the test consumable receptacle and at least one of: a particular type of troponin test (hsCTn or cCTn), a phase at which the test consumable receptacle is to be used, a kit comprising test consumable receptacles, or identifying information of a patient, or a combination of these.

16. The system of claim 15, wherein the tag associates the test consumable receptacle with the initial hsCTn test, the subsequent hsCTn test, the second cCTn test, the third cCTn, the third cCTn test, or the fourth cCTn test.

17.-24. (canceled)

25. A method of instructing a user to determine acute myocardial infarction (AMI) in a subject that presents with acute coronary symptoms, the method comprising:
providing to a user a kit comprising:
a first high sensitivity cardiac troponin test (hsCTn) test comprising:
an initial hsCTn test; and
a subsequent hsCTn test;
a second conventional cardiac troponin (cCTn) test; and
a third cCTn test; and
instructing the user to perform:
the initial hsCTn test on an initial sample obtained from the subject at the time the subject presents with acute coronary symptoms;
the subsequent hsCTn test on a subsequent sample obtained from the subject no more than one hour after the subject presents with acute coronary symptoms, wherein if the first hsCTn test is negative, acute myocardial infarction (AMI) is ruled out; and
if AMI is not ruled out by the subsequent hsCTn test, to perform at least one of:
the second cCTn test on a second sample obtained from the subject at a three-hour phase after the subject presents with acute coronary symptoms; or
the third cCTn test on a third sample obtained from the subject at a four-hour phase after the subject presents with acute coronary symptoms; and
instructing the user to recommend treating the subject for AMI if the second cCTn test or third cCTn test is positive.

26. The method of claim 25, further comprising instructing the user to perform the second cCTn test on the second sample if the first hsCTn test is positive.

27. The method of any one of claims 25-26, further comprising instructing the user to perform the following if the first hsCTn test is observational or if the second cCTn test is observational:
to perform the third hsCTn test on the third sample,
wherein if the third hsCTn test is positive, to perform a third cCTn test on the third sample or on another sample obtained from the subject at the four-hour phase after the subject presents with acute coronary symptoms; and
instructing the user that if the third hsCTn test is not positive, the third cCTn test is not required.

28. The method of claim 27, further comprising instructing the user that if the third hsCTn test is not positive, or if the third cCTn test is not positive, to perform the following:
a fourth cCTn test on a fourth sample obtained from the subject at a six-hour phase after the subject presents with acute coronary symptoms,
wherein if the fourth cCTn test is negative, instructing the user that no AMI treatment is required for the subject, and
wherein if the fourth cCTn test is not negative, instructing the user that the subject remains a candidate for conventional 6-12 hour AMI observation.

29.-32. (canceled)

33. A method of determining acute myocardial infarction (AMI) in a subject who presents with acute coronary symptoms, the method comprising:
performing a first high-sensitivity cardiac troponin (hsCTn) test comprising:
receiving an initial sample obtained from the subject at the time the subject presents with acute coronary symptoms and performing an initial hsCTn test on the initial sample; and
receiving a subsequent sample obtained from the subject at a first phase after the subject presents with acute coronary symptoms and performing a subsequent hsCTn test on the subsequent sample,
wherein if the first hsCTn test is negative, determining an absence of AMI; and
if the first hsCTn test is not negative:
receiving at least one of:
a second sample obtained from the subject at a second phase after the subject presents with acute coronary symptoms; or
a third sample obtained from the subject from the subject at a second phase after the subject presents with acute coronary symptoms; and
performing at least one of:
a second conventional cardiac troponin (cCTn) test on the second sample; or
a third cCTn test on the third sample, wherein if the second cCTn test or third cCTn test is positive, the subject is determined to have AMI.

34. The method of claim 33, wherein if the first hsCTn test is positive, the second cCTn test is performed on the second sample.

35. The method of any one of claims 33-34, wherein if the first hsCTn test is observational or if the second cCTn test is observational, the method further comprises:
performing the third hsCTn test on the third sample,
wherein if the third hsCTn test is positive, a third cCTn test is performed on the third sample or on another sample obtained from the subject at a third phase after the subject presents with acute coronary symptoms, and
wherein if the third hsCTn test is not positive, the third cCTn test is not required.

36. The method of claim 35, wherein if the third hsCTn test is not positive, or if the third cCTn test is not positive, the method further comprises:
performing a fourth cCTn test on a fourth sample obtained from the subject at a six-hour phase after the subject presents with acute coronary symptoms,
wherein if the fourth cCTn test is negative, no AMI treatment is required for the subject, and
wherein if the fourth cCTn test is not negative, the subject remains a candidate for conventional 6-12 hour AMI observation.

37.-46. (canceled)
